(19) United States
(12) Patent Application Publication
(10) Pub. No.: US 2026/0297555 A1
(43) Pub. Date: Oct. 1, 2026

(54) SEMI-SOLID PHASE EXTRACTION OF NUCLEIC ACIDS USING A QUATERNARY SALT AND CATION MIXTURE IN ALKALINE CONDITIONS

(71) Applicant: BIOCARTIS NV, Mechelen (BE)

(72) Inventors: Ludovic BARAULT, Mechelen (BE); Max HAESENDONCKX, Mechelen (BE); Kristof GILLIAERT, Mechelen (BE); Eef VANKERCKHOVEN, Mechelen (BE); Tania IVENS, Mechelen (BE)

(21) Appl. No.: 19/477,677

(22) PCT Filed: Apr. 26, 2024
(86) PCT No.: PCT/EP2024/061528

§ 371 Date: Oct. 22, 2025

(30) Foreign Application Priority Data
Apr. 26, 2023 (NL) 2034698

(51) Int. Cl. C12N 15/10

(52) U.S. Cl. CPC C12N 15/1006

(57) ABSTRACT

The present invention relates to improved methods for purifying nucleic acids comprising quaternary ammonium salts and free cations at alkaline conditions, requiring less, less costly and less noxious materials than extraction methods based on highly chaotropic salts. The method is broadly applicable but 5 especially useful in micro-fluidic cartridges.

### TECHNICAL FIELD

The present invention relates to improved methods for purifying nucleic acids comprising quaternary ammonium salts and free cations at alkaline conditions, requiring less, less costly and less noxious materials than extraction methods based on highly chaotropic salts. The method is broadly applicable but especially useful in micro-fluidic cartridges.

### BACKGROUND

Purification of nucleic acids has a long history, and comes with various methods, but it has revolutionized the field of biology offering the possibility to dissect the genetic information of a cell. Such purification processes rely on three main steps: (1) nucleic acid release from its nucleoprotein complex or from the cell using a lysis step, (2) the purification step itself, allowing the isolation of the nucleic acids from the other molecules from the lysate, and (3) the recovery of the nucleic acids allowing the retrieval of the nucleic acids at working condition, preferably at high purity and high concentration.

Various methods were advanced to address the specific needs and the particular input material. Two main routes of purification processes became popular and were further developed: (i) extraction by phase separation, which includes precipitation and centrifugation steps, and (ii) solid phase extraction (SPE) in which the nucleic acids are bound to a solid phase, generally a silica solid support, followed by washing and elution from the support. In some combination methods steps of phase separation and SPE methods are joined.

Phase separation extraction is cheap, since there is no need for expensive buffers or solid phases. However, it makes use of hazardous compounds, such as phenol and chloroform, and does not provide optimal extraction yields. Hence, phase separation extraction is therefore privileged for applications where the quantity of the input NA (nucleic acid and nucleic acids are both abbreviated as “NA” herein) is not an issue, while securing cost effectiveness, such as, e.g. extraction of nucleic acids from abundantly present material of micro-organisms. Due to the need for centrifugation steps, phase separation extraction is not easily amenable for automation.

Solid phase extraction is normally the preferred alternative for purification of NA from complex and/or valuable samples. SPE typically requires chaotropic salts, but these can inhibit downstream processes such as polymerase amplification if carried into the elution buffer. SPE thus requires extensive washing.

Combination methods have also been developed, mixing steps and/or buffers from SPE and phase separation protocols. Extraction protocols using quaternary ammonium salts are examples of combination methods. The latter combination methods can be used for a wide variety of input material, but are predominantly used for purifying high-molecular weight DNA molecules from complex plant material, comprising high contents of polysaccharides. In these protocols, the quaternary ammonium salts are used exclusively as a detergent in the NA release step at alkaline pH, when lysing the cells and denaturing the proteins and polysaccharides of the input material. For instance, Huang and colleagues describe a procedure, which combines the effects of the high DNA yield of complex starting materials using quaternary ammonium salts with the high DNA purity via SPE using a silica matrix (Huang et al., 2000 BioTechniques 28:432-434). However, the use of quaternary ammonium salts complicates the next purification step, since it involves a precipitation step of salt and proteins as well as using chloroform and phenol, which are hazardous and non-compatible with lab-on-a-chip (LoC) devices. The procedure also involves a separation of the hydrophilic phase containing the DNA from the chloroform phase, which step is laborious by itself, but has also a high risk of contamination, impairing the efficiency of the downstream enzymatic processes.

CN115141832 describes a purification process of DNA from stool samples, in which the lysis step uses CTAB as a detergent, but comprises in addition extreme alkaline pH conditions, EDTA and glass beads. In this process no chloroform or phenol was used, but required lowering the pH and multiple, extensive washing steps via spinning columns.

Thus, although the phase separation and combination methods enable purification of a wide variety of input samples in a cost-effective manner, the drawbacks of being laborious, not apt to automation, frequently the low yield, extensive washing, and/or the presence of inhibitory and hazardous components override the advantages.

LoC devices or disposable cartridges mainly make use of solid phase extraction. Micro-fluidic (lab-on-a-chip) PCR array cartridges are very versatile and are used in a growing number of diagnostic assays. These micro-fluidic cartridges are part of a user-friendly, autonomic operational platform, such as the Idylla™ platform by Biocartis. The standard micro-fluidic cartridges allow NA extraction, followed by purification, amplification of NA and automatic detection. The user only has to collect the sample, inject the raw sample into the cartridge, insert the cartridge into the instrument, which automatically supports micro-fluidics within the cartridge, and then press the “start” button. This automated and contained process flow protects the sample from contamination and minimizes the user's exposure to the sample. For efficiency reasons the micro-fluidic cartridges are standardized as far as possible and manage a host of very diverse samples ranging from solid Formalin Fixed Paraffin Embedded (FFPE) samples, biopsies, cancerous tissue, and stool to liquid samples. The methods employed in the cartridges should preferably enable the purification of nucleic acids from all of these diverse sample types.

In general, after NA release, the micro-fluidic cartridges employ SPE purification as a convenient method, avoiding many of the problems associated with e.g. phase separation extraction (cf. liquid-liquid purification). The SPE purification involves 3 key steps: (i) nucleic acid binding to the solid phase, (ii) washing, and (iii) elution of the purified nucleic acids.

Due to its high performance allowing NA extraction yields of >50% from biological samples, the Boom protocol using for the first time silica-based nucleic acid purification technology (see EP389063) has quickly become the gold standard and is widely used, e.g. QIAGEN's QIAamp Circulating Nucleic Acid kit.

Various studies indicate that the binding of NA to the solid matrix in the purification process depends mainly on the pH and salt content of the buffer, in which the main principle governing the absorption process is hydrogen-binding interaction of the NA with the hydrophilic matrix under chaotropic conditions (see e.g. Vandeventer et al., 2012, J. Phys. Chem. B., 116:5661-5670).

In the Boom and related protocols, the use of highly chaotropic compounds, such as GuSCN and GuHCl, is used for both the NA release step as well as the purification step. The low pH and presence of chaotropic salts not only facilitate the DNA isolation but also serve an additional purpose, by lysing cells and protecting DNA from degradation by denaturing nucleases. The chaotropic compounds also act on NA by removing their water shell, and thus increasing their affinity for the silica membrane. Alcohol is generally added to the mixture, favouring the dehydration of NA. Without its water shell, the phosphate backbone of the NA will be able to form direct hydrogen bonds with the protonated silica membrane under acidic conditions. At higher pH, the abundance of guanidine cations can shield the electrostatic forces between the negatively charged phosphate backbone and the negatively charged silanol groups. Both effects may promote hydrophobic interaction between the bases and the siloxane bridges, thus enabling adsorption of nucleic acids to the silica membrane.

The bound NA are subsequently washed carefully with highly concentrated alcohols (often 70%-97% ethanol). This washing procedure should ensure the removal of any residual inhibitory compounds originating from the biological sample or the binding buffer comprising the chaotropic compound.

Finally, the NA are eluted with a low ionic strength solution at neutral or slightly basic pH.

However, the Boom and related protocols require large volumes of binding buffer and expensive, strong and at high concentration chaotropic salts. In addition, the chaotropic salts are corrosive and can cause severe skin burns and eye damage, and are harmful if swallowed, in contact with skin or if inhaled. Very toxic gases are released when these chaotropic salts react with acids or bleaches. Moreover, the chaotropic salts inhibit polymerases used in downstream target applications, such as polymerase chain reaction (PCR) and NGS (next generation sequencing). As a consequence, SPE approaches using chaotropic salts require meticulously executed safety measures and rigorous, cumbersome, and lengthy wash steps.

A further complicating factor is that the samples generally have minute and variable amounts of target nucleic acids, which are embedded in vast quantities of excess non-target NA and other contaminants. Inevitably, the amount of the target NA that can be subject to detection is diminished additionally since every step of processing the raw sample for final detection comes with a loss of material. On the other hand, the eventual quantity and quality of the target NA is a main determinant of the robustness of an assay. Hence, increasing the input quantity of the raw sample, and thus of the target material would augment the robustness of an assay. Although bench extraction is not limited in terms of volume, the ratio sample/binding buffer (BB) required in Boom-based protocols results in huge amounts of waste chemicals generated. On the other hand, cartridges have fixed dimensions, because of which they can only contain set volumes of binding buffers, washing solutions, etc., necessary for purifying and processing the target NA. Moreover, the cartridges have preset processing chambers, which consequently limits the amount of the sample that can be dispensed into the cartridge. A further trend is miniaturizing the micro-fluidic cartridges, which reduces the costs-of-goods (CoG), but also limits the amount of sample and thus target NA that can be purified and processed.

Apart from the above curbs, the ingredients for purification and processing must also be compatible with safety standards, omitting forbidden hazardous or noxious materials, as well as being compatible with the fabrics of the cartridge, which further limits the possible compounds that can be used.

Hence, there is an intrinsic conflict between the requirements for purifying and processing the sample and the characteristics of the cartridge.

There remains a need for improving the purification process of NA. Indeed, as mentioned, it seems difficult, if not impossible, to establish a procedure which can be universally applied to point-of-care diagnostics at present (cf. Nasir, et al. 2017 Biomed Res Int. doi: 10.1155/2017/9306564).

A solution was apparently provided by chaotrope-free silica-based NA purification. Notable examples include the-similar-methods published by Hourfar et al. in 2005, Lee et al. in 2008, Johns Hopkins University (WO2016/073824) and MiDiagnostics (WO2018/156906), which all describe the purification of RNA using acidic conditions and kosmotropic salts. However, these disclosures seem to be RNA specific and not suitable for DNA purification in adequate quantity and/or quality.

The patent application WO2021/023854 by Biocartis NV addressed the shortcomings of the chaotrope-free NA-extraction methods by successfully employing a salt comprising a small quaternary organic compound cation at an acidic pH value between 3 and 6 for its kosmotropic properties. The improved purification protocol was extended by a perfected NA-extraction protocol using a quaternary ammonium compound detergent, also at acidic conditions. The method of WO2021/023854 resulted in a better binding buffer to sample volume ratio, thus enabling an increased sample input, less costs and less inhibitory compounds compared to the above-described methods, thus being highly beneficial in fully integrated molecular diagnostic devices.

Nonetheless, there is room for further improvement, such as, for instance, a further improved ratio sample versus binding buffer, hazard reduction, improved yield, less waste, and improved turnaround time (TAT).

The present inventors rethought and redesigned the nucleic acid purification process to be used in micro-fluidic cartridges, but which turned out to be useful in a broad array of applications.

### SUMMARY

The present invention provides a novel, efficient nucleic acid purification protocol. In particular, a purification chemistry is provided that mediates complexing of nucleic acids to silica by using nothing more than a quaternary ammonium salt (QUATS) and a free cation, at a pH between 7-9. The disclosed methods substantially reduce the complexing buffer volume relative to the sample volume, thus enabling an increased sample input, which is highly beneficial in fully integrated molecular diagnostic devices. In addition, the basic pH range of the invention precludes DNA degradation commonly observed with more acidic pH ranges. Since the new method requires fewer washing steps, the TAT is also improved.

Furthermore, the disclosed isolation method provides higher nucleic acid yields than the strong chaotrope-based protocols, such as the Boom protocol. The new method results additionally in less waste and in hazard reduction.

In particular, the present invention provides a method for purifying nucleic acids from a mixture, such as a solution, comprising nucleic acids, the method comprising:
(i) complexing the nucleic acids in the mixture, such as a solution, to a silica support in the presence of a complexing buffer;
(ii) washing the nucleic acids of step (i) with an alcohol; and
(iii) eluting the nucleic acids of step (ii) with an elution buffer;
by which the nucleic acids are purified, wherein the complexing buffer comprises QUATS, a free cation, and has a pH between 7-9, wherein the QUATS is preferably a quaternary ammonium cation having a nitrogen atom at the center and being represented by formula (I)

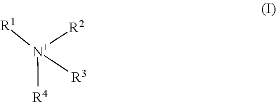

preferably wherein each of R¹, R² and R³ are independently methyl groups (—CH₃) groups, and wherein R⁴ is an alkyl group (CₙH₂ₙ₊₁), with various alkyl chain lengths, preferably even-numbered alkyl chain lengths, even more preferably n=8, 10, 12, 14, 16 or 18, and preferably, wherein the counter-anion of the QUATS is Cl⁻ or Br⁻, even more preferably, wherein the QUATS is chosen from the group consisting of octyl-trimethyl-ammonium bromide (OTAB), octyl-trimethyl-ammonium chloride (OTAC), decyl-trimethyl-ammonium bromide (DTAB), decyl-trimethyl-ammonium chloride (DTAC), dodecyl-trimethyl-ammonium bromide (DoTAB), dodecyl-trimethyl-ammonium chloride (DoTAC), tetradecyl-trimethyl-ammonium bromide (TTAB), tetradecyl-trimethyl-ammonium chloride (TTAC), hexacetyl-trimethyl-ammonium bromide (CTAB), hexacetyl-trimethyl-ammonium chloride (CTAC), octadecyl-trimethyl-ammonium bromide (OctMAB), and octadecyl-trimethyl-ammonium chloride (OctMAC), more preferably chosen from OTAB, OTAC, DTAB, DTAC, DoTAB and DoTAC; and wherein the free cation is preferably chosen from the group consisting of Mg²⁺, Mn²⁺, Li⁺, Na⁺, Zn²⁺, K⁺ and Ca²⁺, more preferably Mg²⁺, Na⁺ or K⁺ and even more preferably the free cation is Mg²⁺; and wherein the pH is preferably between 7.2 and 8.8, or between 7.5 and 8.5 or even between 7.7 and 8.3, even more preferably the pH is about 8; and wherein the silica support is preferably chosen from the group consisting of glass particles, glass powder, silica particles, glass microfibers, diatomaceous earth, silica sand, silica gel, silicon carbide (SiC) and mixtures thereof, or wherein the silica support is chosen from silica membranes, silica impregnated or coated filters, and silica coated magnetic beads.

The present invention provides a method for purifying nucleic acids from a mixture, such as a solution, as described herein, wherein the complexing buffer comprises QUATS in a concentration (mass/vol) between 0.5% to 7%, preferably between 0.75% to 5%, between 0.9% to 4% or between 1% to 3%, or between 1.1% and 2.3%, for instance in a concentration (mass/vol) of about 0.5%, 0.6%, 0.7%, 0.75%, 0.8%, 0.9%, 1.0%, 1.1%, 1.25%, 1.5%, 1.75%, 2%, 2.3%, 2.5%, 3%, 3.5%, 4%, 4.5%, 4.6%, 5%, 5.5%, 6%, 6.5% or 7%; and/or the complexing buffer comprises free cations of the invention in a concentration between 50 mM and 0.5 M, or between 75 mM and 450 mM, or between 100 mM and 400 mM, between 150 mM and 350 mM, preferably between 175 mM and 325 mM, such as between 200 mM and 300 mM, or 225 mM and 275 mM, or most preferably about 250 mM, for instance in a concentration of about 50 mM, about 100 mM, about 150 mM, about 175 mM, about 200 mM, about 225 mM, about 250 mM, about 275 mM, about 300 mM, about 325 mM, about 350 mM, about 400 mM, about 450 mM, or about 0.5 M; and/or wherein the alcohol for washing is chosen from methanol, propan-1-ol, isobutanol, sec-butanol, tert-butyl-alcohol, ethanol, butanol and isopropanol, preferably ethanol, butanol and isopropanol, even more preferably ethanol, preferably, wherein the alcohol is present in concentration of between 70% to 100%, such as 78% to 99%, 80% to 98%, 85% to 97%, such as 90% to 96%, such as, for instance, in concentration of about 70%, about 75%, about 80%, about 85%, about 90%, about 92%, about 95%, about 96%, or about 99%, preferably about 96%; and/or wherein the elution buffer has a pH between 8 and 9, such as between 8.1 and 8.7 or more preferably between 8.3 and 8.6, even more preferably wherein the elution buffer has a pH of about 8.4, such as, for instance, wherein the elution buffer has a pH of about 8, about 8.1, about 8.3, about 8.4, about 8.6, about 8.7 or about 9; preferably the elution buffer is chosen from: Tris-HCl at a concentration between 0.01 M and 0.05 M, such as between 0.02 M and 0.04 M, preferably about 32 mM, at a pH between 8 to pH 9, preferably a pH of about 8.4; MgCl₂ at a concentration between 3 mM and 11 mM, such between 4 mM and 8 mM, preferably about 4.6 mM; and KCl at a concentration between 40 mM to 150 mM, such as between 60 mM and 120 mM, preferably about 96 mM.

The present invention provides a method for purifying nucleic acids from a mixture, such as a solution, as described herein, wherein the solution is a tissue sample, such as a biopsy sample, preferably, said tissue sample is a solid tissue sample, for example, the tissue sample is from breast, lung, colon, rectum, brain, heart, liver, spleen, thyroid, thymus, kidney, testis, ovary, tumors, tissue biopsies, or wherein the tissue sample is a liquid tissue sample, for example, blood or any blood constituents, such as white blood cells, serum, plasma, bodily fluids such as bone marrow aspirates, cerebral spinal fluid, amniotic fluid, lung pleural effusion, peritoneal fluid, interstitial fluid, saliva, sweat, tears, breast milk, semen, stool, pleural fluid, or a suspension of cells and other body fluids such as derived from washings, etc.

The present invention provides a method for purifying nucleic acids from a mixture, such as a solution, as described herein, wherein the nucleic acid is DNA, preferably wherein the DNA is cell free DNA or circulating tumor DNA, and/or wherein the NA is preferably purified to at least 60% purity, such as at least 70%, at least 75%, at least 80%, at least 85%, at least 90%, at least 95%, at least 97%, such as at least 99% purity, e.g. >99.5%, ≥99.9%, or even ≥99.95%.

The present invention provides a method for purifying nucleic acids from a mixture, such as a solution, as described herein, wherein the method is preceded by a lysis step, preferably said lysis is chosen from mechanical lysis, thermal lysis, chemical lysis, biochemical lysis or electrical lysis, and/or preceded by a protease treatment, preferably a protease K treatment, and/or preceded by a liquefaction step.

The present invention provides a method for purifying nucleic acids from a mixture, such as a solution, as described herein, wherein the method is performed inside of a cartridge, preferably being a fluidic cartridge. Preferably, the cartridge configured to purify nucleic acids from a mixture such as a solution, according to the invention comprises at least 3 containers for storing liquids, at least a sample chamber adapted for receiving a sample, and a processing chamber comprising a silica solid support, wherein containers are in fluid connection with the sample chamber and/or the processing chamber, and wherein at least one container containing a complexing buffer; at least one container containing an alcohol; and at least one container containing an elution buffer; wherein the complexing buffer comprises QUATS and free cations and has a pH between 7-9, wherein the QUATS having a nitrogen atom at the center is represented by formula (I), wherein each of R¹, R² and R³ are independently methyl groups (—CH₃) groups, and wherein R⁴ is an alkyl group (CₙH₂ₙ₊₁), with various even-numbered alkyl chain lengths, preferably n=8, 10, 12, 14, 16 or 18, and wherein the counter-anion is Cl⁻ or Br⁻.

The present invention provides the use of the method as described herein or the cartridge as described herein for isolating NA from a mixture, such as a solution.

### BRIEF DESCRIPTION OF THE FIGURES

For a further understanding, reference is made to the following detailed description taken in conjunction with the accompanying figures, in which:

FIG. 1: shows exemplary quaternary ammonium cations of formula (I), wherein each of R¹, R² and R³ are independently methyl groups (—CH₃) groups, and wherein R⁴ is an alkyl group (CₙH₂ₙ₊₁), with an even-numbered alkyl chain length, selected from the group consisting of n=8, 10, 12, 14, 16 and 18, and wherein the counter anion is Br⁻.

FIG. 2: shows a box plot diagram of a comparison of the QUATS-process of the invention with the classic Guanidinium (GU) based extraction process. The Y-axis displays Cq values distribution of matched samples extracted with both GU-based extraction protocol (Gu) or the QUATS-process (using CTAB). The X-axis displays different targets: KRAS G12V, EGFR C797S, EGFR L858R, HER2 and KIF11. The QUATS-process used 20 samples, the GU-based extraction protocol used 12 samples.

FIG. 3: shows the results of fragment analyzer of a sample comprising a spiked DNA ladder extracted with different complexing buffers: (A) 2.3% CTAB; (B) 2.3% CTAC; (C) 4.6% CTAC; and (D) 2.3% CTAB/4.6% CTAC.

FIG. 4: shows a box plot diagram comparing the QUATS-process using 1.1% DoTAC with the classic GU-based extraction process. The Y-axis displays Cq values distribution of matched samples extracted with both GU-based extraction protocol (Gu) or the QUATS-process (DoTAC). The X-axis displays different targets: EGFR C797S, KRAS G12V, HER2, KIF11 and EGFR L858R.

FIG. 5: shows a box plot diagram comparing the QUATS-process using various QUATS with differing aliphatic tails and counter ions: DoTAC (1.1%), CTAC (2.3%), CTAB (2.3%), DoTAB (2.3%), and positive control. The Y-axis displays Cq values distribution of matched samples extracted with the QUATS-process using the various QUATS. The X-axis displays different targets: EGFR C797S, KRAS G12V, HER2, KIF11 and EGFR L858R.

FIG. 6: relates to the di-aliphatic chain didecyldimethylammonium bromide (DDAB). FIG. 6A shows the chemical structure of DDAB. FIG. 6B shows a phase separation when adding DDAB to 50 mM TRIS-HCl buffer pH 8, 250 mM of MgCl₂ and 5% butanol in manufacturing a complexing buffer.

FIG. 7: shows photographs of the QUATS-process on Macherey-Nagel columns using complexing buffers consisting of DoTAC (at 1.1%) dissolved in a 0.05 M Tris-HCl pH 8 buffer and 5% butanol with and without free cations. FIG. 7A shows side- and top-view photographs of the columns with the complexing buffer with 250 mM Mg²⁺ (+MgCl₂) and without Mg²⁺ (—MgCl₂). FIG. 7B shows columns containing the complexing buffer (CB) with a concentration range of free cations: 0 mM, 50 mM, 125 mM and 250 mM.

FIG. 8: shows the effect of varying concentrations of free cations on the purification process: 0 mM, 100 mM, 200 mM, 300 mM, 400 mM, and positive control. The complexing buffer consisted further of CTAB (at 2.3%) dissolved in a 0.05 M Tris-HCl pH 8 buffer and 5% butanol. 2000 copies of the target sequences EGFR C797S, KRAS G12C, EGFR G719A, HER2 were spiked in 1 ml plasma sample. The target sequences including the native KIF11 gene were processed according to standard conditions and subjected to qPCR. In FIG. 8A, the Background versus Corrected Cycle is represented. FIG. 8B displays Cq values of all five amplicons.

FIG. 9: shows the effect of varying concentrations of free cations (Mg²⁺) on the QUATS-process: 0 mM, 50 mM, 125 mM, 250 mM, and positive control. The complexing buffer consisted further of DoTAC (at 1.1%) dissolved in a 0.05 M Tris-HCl pH 8 buffer and 5% butanol. 2000 copies of the target sequences EGFR C797S, KRAS G12V, HER2, and EGFR L858R were spiked in 4 ml plasma sample. The samples including the native KIF11, were processed according to standard conditions and subjected to qPCR. In FIG. 9A, the Background vs corrected cycle is represented. FIG. 9B displays Cq values of all five amplicons.

FIG. 10: shows the effect of free cations on the QUATS-process. The Y-axis displays Cq values of all five amplicons: native KIF11, while 2300 copies of the target sequences KRAS G12C, EGFR C797S, EGFR G719A and HER2 were spiked in the samples. The X-axis displays different binding buffer compositions: no ions corresponding to the complexing buffer containing DoTAC (at 1.1%) dissolved in a 0.05 M Tris-HCl pH 8 buffer, 5% butanol, and supplemented with Mg²⁺, K⁺, Li⁺, Na⁺, and Ca²⁺, or no free cations. All free cations were supplemented at a concentration of 250 mM (FIG. 10A). The median Cq values are summarized in FIG. 10B.

FIG. 11: shows the effect of butanol in the complexing buffer of the QUATS-process: complexing buffer without 5% butanol, complexing buffer with 5% butanol. The complexing buffer consisted further of DoTAC (at 1.1%) dissolved in a 0.05 M Tris-HCl pH 8 buffer and 250 mM MgCl₂. 2300 copies of the target sequences EGFR C797S, KRAS G12V, HER2 and EGFR L858R were spiked in 4 ml plasma sample. The samples, including native KIF11, were processed according to standard conditions and subjected to qPCR. In FIG. 11A, the Background versus Corrected Cycle is represented. FIG. 11B displays the Cq values of all five amplicons.

FIG. 12: shows the effect of the pH of the complexing buffer on the QUATS-process: the complexing buffer containing CTAB (at 2.3%) in a 0.05 M Tris-HCl, with different pH's: pH 7, pH 8 and pH 9. The pH effects were tested on 5 different targets: native KIF11, and 2300 copies of the target sequences EGFR C797S, KRAS G12C, EGFR G719A and HER2 spiked in the samples. The samples were processed according to standard conditions and subjected to qPCR. FIG. 12A: the X-axis displays the number of corrected cycles, the Y-axis displays Offset drift (corrected). FIG. 12B displays the Cq values of all five amplicons.

FIG. 13: shows the effect of washing steps in the QUATS-process. After complexing different NA to the silica support with a complexing buffer containing DoTAC (at 1.1%) dissolved in a 0.05 M Tris-HCl pH 8 buffer, 5% butanol and 250 mM MgCl₂, NA were subjected to wash steps before assaying: no wash step; 1 wash step; 2 wash steps; 3 wash steps; and positive control. 2000 copies of the target sequences EGFR C797S, KRAS G12V, HER2 and EGFR L858R were spiked in 4 ml plasma sample. The samples, including native KIF11, were processed according to standard conditions and subjected to qPCR. FIG. 13A: the Background vs corrected cycle is represented. FIG. 13B displays the Cq values of all five amplicons.

FIG. 14: shows the effect of pH and magnesium concentration in the elution buffer on the QUATS-process. A complexing buffer with DoTAC 1.1% at pH 8 and one wash with ethanol 96% was used, but the QUATS-process was varied by the use of six different types of elution buffers. The sequences detected were EGFR G797S, KRAS G12V, HER2, KIF11 and EGFR L858R. Except for native KIF11, 2300 copies were spiked into a sample. In FIG. 14A, an elution buffer containing 3.8 mM of Mg²⁺ was tested at pH 8, pH 8.2, and pH 8.5. In FIG. 14B, an elution buffer containing 4.6 mM of Mg²⁺ was tested at pH 8, pH 8.2, and pH 8.5. The Cq values of all five amplicons are displayed.

### DETAILED DESCRIPTION

Exemplary embodiments according to aspects of the present invention may satisfy one or more of the above-mentioned desirable features set forth above. Other features and advantages will become apparent from the detailed description which follows.

In this application, the use of the singular includes the plural unless specifically stated otherwise. In this application, the use of “or” means “and/or” unless stated otherwise. Furthermore, the use of the terms “including” and “comprising”, as well as other forms, such as “includes”, “comprises”, “included” and “comprised”, is not limiting.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose. In the event that one or more of the incorporated literature and similar materials differs from or contradicts this application, including but not limited to defined terms, term usage, described techniques, or the like, this application controls.

The inventors have developed a novel nucleic acid purification chemistry. This chemistry is at least as effective as the current gold standard, i.e. the Boom protocol, but requires less, less costly, and less hazardous materials. The present invention effectively switched the ratio of sample<extraction buffers according to the contemporaneous Boom-protocols to sample>extraction buffers according to the method of the present invention. In addition, the new method is wholly suitable for automation. This makes the present chemistry very suitable for LoC applications, with its small dimensions, and availability of only minute sample input. In particular, the present invention provides a method for purifying nucleic acids from a mixture, such as a solution, comprising nucleic acids, the method comprising:
(i) complexing the nucleic acids in the liquid sample to a silica support in the presence of a complexing buffer;
(ii) washing the nucleic acids of step (i), i.e. the nucleic acids complexed to the silica support in the presence of QUATS and a free cation, with an alcohol;
(iii) eluting the nucleic acids of step (ii), i.e. the nucleic acids that have been washed, with an elution buffer;
   by which the nucleic acids are purified, wherein the complexing buffer comprises quaternary ammonium salts (QUATS) and a free cation, and has a pH between 7-9. This method is also referred to as “QUATS-process” herein.

The present inventors rethought and redesigned the NA purification process to be used in micro-fluidic cartridges, but eventually the method turned out to be useful in a broad array of applications.

Without being bound by theory, the present inventors hypothesized that particular quaternary ammonium salts form micelles in a polar environment. At slightly alkaline conditions, the resulting micelles will have a hydrophobic core and the positive charge is located at the outside of the micelles. The positive charge on the outside of the micelles complex the nucleic acids and the silica membrane by on the one hand trapping the negatively charged nucleic acids and on the other hand by being attracted to the negatively charged silica membrane. The micelles effectively liaise between the nucleic acids and silica membrane.

The silica surface charge state of the silica support and the types of surface silanol groups present are predominantly dictated by the silica's point of zero charge, which can vary from 1.5 to 3.6. In most cases, the silica surface is negatively charged (neutral to weakly at pH<5, and more strongly at higher pH). The present inventors hypothesized that interactions between the components of the invention, e.g. nucleic acids and QUATS, and the silica support are modulated by the charge (siloxane (Si—O—Si) bridges) as a function of electrolyte concentration. The present free cations will interact indirectly with the deprotonated backbone and the major groove of the nucleic acid facilitating also nucleic acid compaction.

Washing the complex of nucleic acids-micelles-silica support with a high percentage alcohol, such as, e.g., 70% alcohol or more, results in removal of the contaminants but also bursting of the micelles and removal of the remaining QUATS. Completely counterintuitively, the negatively charged nucleic acids remain or become complexed to the silica support. It was believed that this may be due to dehydration of the nucleic acids, which would in turn favorize adsorption to the silica support. The process is probably augmented by the presence of free cations, which are expected to be insoluble in the presence of alcohol and which may act as a bridge between the silica membrane and the nucleic acids.

Next, the elution buffer rehydrates the nucleic acids, which are subsequently released from the silica support. The resulting nucleic acids in elution buffer are consequently purified and can be used for further processing.

The inventors determined that the particular quaternary ammonium salts of the present invention would have the ability to form micelles in a polar environment, in which the core is hydrophobic, the palisade layer of the QUATS is located on the inside of the micelles and the positively charged ammonium headgroup is located at the outside of the micelle (cationic micelle). The micelles of the invention form a complex between and with the negatively charged nucleic acids and the silica support. Subsequently, while the nucleic acids retain complexed with the silica support, impurities and contaminants are washed away, and after elution the nucleic acids are isolated from the liquid sample.

In the present context, a “micelle” is an aggregate of surfactant amphipathic lipid molecules, preferably the QUATS of the invention, dispersed in a liquid, forming a colloidal suspension. The micelles of the invention are not limited to a particular shape, but preferred shapes are (approximately) spherical, ellipsoids, cylinders, and bilayers. The person skilled in the art will appreciate that the shape and size of a micelle are a function of the molecular geometry of its surfactant molecules and solution conditions, such as, for instance, surfactant concentration, temperature, pH, and ionic strength. Within the limits of the invention, the skilled artisan can configure the process of forming micelles (micellisation) according to the specific needs.

In the present invention, the term “complexing” in the context of complexing nucleic acids to a solid support is not intended to refer to any intermolecular force between the quaternary ammonium salt, the nucleic acid and the silica support specifically, but encompasses any force potentially enabling the interaction between the quaternary ammonium salt, the nucleic acid and/or the silica support, such as, for instance, ionic forces, hydrogen bonding, Van Der Waals dipole-dipole interactions or Van Der Waals dispersion forces (“London dispersion” forces). In this regard, the term “complexing” in the context of complexing nucleic acids to a solid support is not intended to refer to any order of interaction, but encompasses the direct interaction between the quaternary ammonium salt and the nucleic acid, the direct interaction between the quaternary ammonium salt and the silica support, the direct interaction between the nucleic acid and the silica support, the direct interaction between the nucleic acid and both the silica support and the quaternary ammonium salt, the direct interaction between the quaternary ammonium salt and both the nucleic acid and the silica support, the direct interaction between the silica support and both the nucleic acid and the quaternary ammonium salt, as well as all components interacting with each other directly. The inventors contemplated that combining the QUATS with the free cation would save valuable space in e.g., a cartridge. The complexing buffer comprising the quaternary ammonium salt with the free cation at pH 7-9 was stable over prolonged time intervals and apt to isolate the nucleic acids in the method according to the invention. In fact, it came by a surprise that the QUATS-process of the invention worked so efficiently at pH 7-9, considering that contemporaneous elution protocols releasing the bound nucleic acids from the silica supports normally require the use of Tris-HCl pH 8 buffers.

Hence, in an embodiment, the present invention provides a complexing buffer comprising quaternary ammonium salt (QUATS) and a free cation, at a pH between 7-9.

In a preferred embodiment, the quaternary ammonium salts comprise a quaternary ammonium cation having a nitrogen atom at the center and being represented by formula (I)

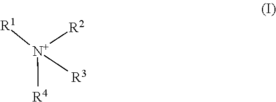

Alternatively, said quaternary ammonium cation having a nitrogen atom, may herein be represented as [R⁴N]⁺, wherein R⁴ represents the presence of 4 independent R groups, defined as R¹, R², R³ and R⁴. In an embodiment, the positively charged atom of the QUATS is nitrogen.

In a preferred embodiment, a method for isolating nucleic acids from a mixture, such as a solution, comprising nucleic acids according to the invention is provided, wherein the corresponding anion of the QUATS is Cl⁻ or Br⁻, preferably Cl⁻.

In a preferred embodiment, a method according to the invention is provided, wherein each of R¹, R² and R³ are independently methyl groups (—CH₃) groups, and wherein R⁴ is an alkyl group (CₙH₂ₙ₊₁), with various alkyl chain lengths, preferably various even-numbered alkyl chain lengths, even more preferably n=8, 10, 12, 14, 16 or 18, more preferably, n=8, 10, 12, and even more preferably n=12.

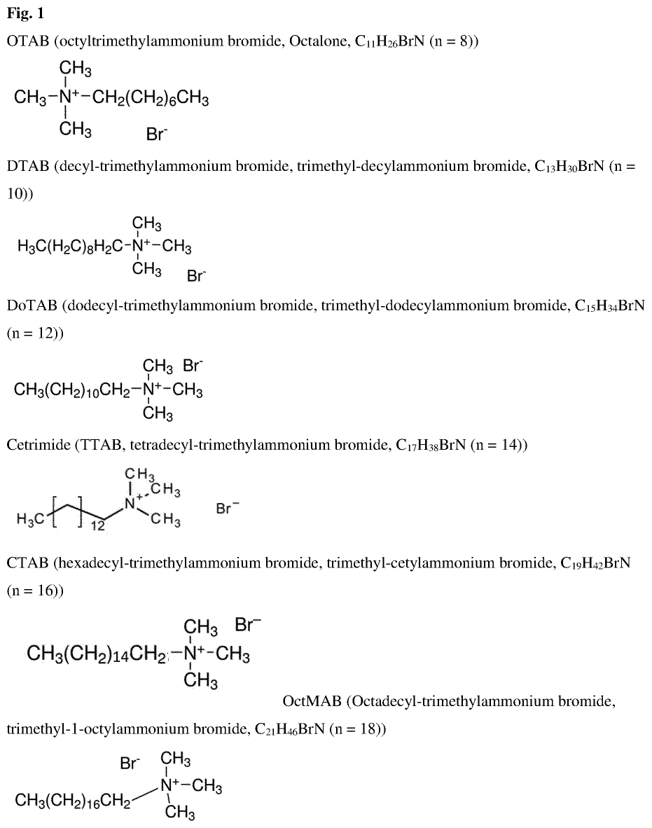

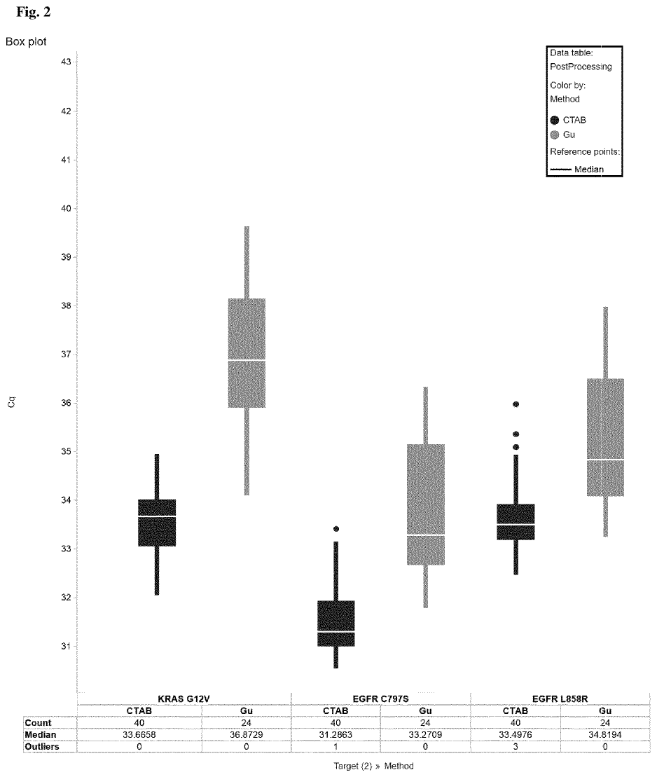

In a preferred embodiment, the quaternary ammonium salts are quaternary ammonium cations [R⁴N]⁺, wherein each of R¹, R² and R³ are independently methyl groups (—CH₃) groups, and wherein R⁴ is an alkyl group (CₙH₂ₙ₊₁), with an even-numbered alkyl chain length, selected from the group consisting of n=8, 10, 12, 14, 16 and 18, and wherein the corresponding anion is Cl⁻ or Br⁻. Exemplary alkyl chain length QUATS according to the invention are shown in FIG. 1. In this figure, Br⁻ is indicated as counter-ion but as detailed below, the exemplary alkyl chain length QUATS according to the invention can have other counter-ions, such as Cl⁻ as counter-ion, as well.

It was found that QUATS, in which R⁴ is an alkyl group containing 8, 10 or 12 hydrocarbons (n=8 or 10 or 12) performed even better than QUATS wherein R⁴ is an alkyl group containing more than 12 hydrocarbons (n>12). It was hypothesized that a smaller alkyl chain may facilitate compaction of the micelles, and consequently increase the surface area to complex. This would also predict that the QUATS with smaller alkyl chains could be present in lesser concentration than with larger alkyl chains, while performing the same. This was confirmed experimentally, in which CTAB performs better at higher percentages (≥2.3%) while DoTAB or DoTAC perform equally well at 1.1% and 2.3%. Hence, in an embodiment, a method is provided, wherein n=8, 10 or 12, most preferably n=12.

In a preferred embodiment, a method for isolating nucleic acids from a mixture, such as solution, comprising nucleic acids according to the invention is provided, wherein the QUATS is chosen from the group consisting of
octyl-trimethyl-ammonium bromide (OTAB);
octyl-trimethyl-ammonium chloride (OTAC);
decyl-trimethyl-ammonium bromide (DTAB);
decyl-trimethyl-ammonium chloride (DTAC);
dodecyl-trimethyl-ammonium bromide (DoTAB);
dodecyl-trimethyl-ammonium chloride (DoTAC);
tetradecyl-trimethyl-ammonium bromide (TTAB);
tetradecyl-trimethyl-ammonium chloride (TTAC);
hexadecyl-trimethyl-ammonium bromide (CTAB);
hexacetyl-trimethyl-ammonium chloride (CTAC);
octadecyl-trimethyl-ammonium bromide (OctMAB); and
octadecyl-trimethyl-ammonium chloride (OctMAC).

In an embodiment, a method for isolating nucleic acids from a mixture, such as solution, comprising nucleic acids according to the invention is provided, wherein the complexing buffer comprises QUATS in a concentration (mass/vol) between 0.5% to 7%, preferably between 0.75% to 5%, between 0.9% to 4% or between 1% to 3%, or between 1.1% and 2.3%. In a preferred embodiment, a method is provided, wherein the complexing buffer comprises QUATS in a concentration (mass/vol) of about 0.5%, 0.6%, 0.7%, 0.75%, 0.8%, 0.9%, 1.0%, 1.1%, 1.25%, 1.5%, 1.75%, 2%, 2.3%, 2.5%, 3%, 3.5%, 4%, 4.5%, 4.6%, 5%, 5.5%, 6%, 6.5%, 6.9% or 7%. In a further preferred embodiment, a method is provided, wherein the complexing buffer comprises CTAB in a concentration of about 2.3% (mass/vol), wherein the complexing buffer comprises DoTAB (n=12) in concentration of about 1.1% (mass/vol) or wherein the complexing buffer comprises DoTAC (n=12) in a concentration of about 1.1% (mass/vol).

In an embodiment, a method according to the invention is provided, wherein the pH is slightly basic, such as a pH between 6.95 and 9.1, preferably a pH between 7 and 9, more preferably a pH between 7.2 and 8.8, or between 7.5 and 8.5 or even between 7.7 and 8.3, even more preferably the pH is about 8.

Although not necessary for the invention per se, it has been observed that the addition of free cations in the method of the invention facilitates the purification process, increasing the solubility of the QUATS and sample, which facilitates the flow through. This would be more valuable for LoC applications than for purification “on the bench”. It is believed that the free cations may interact indirectly with the deprotonated phosphate backbone and major groove of the nucleic acid. The cations would form a cation bridge between the silica support and nucleic acids, improving the adsorption of the nucleic acids, and hence increase the overall extraction efficiency.

Accordingly, in an embodiment, a method according to the invention is provided, wherein the free cation is chosen from the group consisting of Mg²⁺, Mn²⁺, Li⁺, K⁺, Na⁺, Zn²⁺ and Ca²⁺, more preferably Mg²⁺, Na⁺ and K⁺ and even more preferably the free cation is Mg²⁺. A further advantage of using free cations, and especially Mg²⁺, is their ability to solubilize proteins and increasing the fluidity of the sample, and to increase the affinity to the silica support. Similar to the preferred anions of the QUATS, the preferred anion is chosen from the group consisting of Cl⁻ and Br⁻, preferably Cl⁻.

In an embodiment, a method for purifying nucleic acids from a mixture, such as a solution, comprising nucleic acids according to the invention is provided, wherein the complexing buffer comprises free cations of the invention in a concentration between 50 mM and 0.5 M, or between 75 mM and 450 mM, or between 100 mM and 400 mM, between 150 mM and 350 mM, preferably between 175 mM and 325 mM, such as between 200 mM and 300 mM, or 225 mM and 275 mM, or most preferably about 250 mM. In a preferred embodiment, a method is provided, wherein the complexing buffer comprises free cations of the invention in a concentration of about 20 mM, about 25 mM, about 50 mM, about 100 mM, about 150 mM, about 175 mM, about 200 mM, about 225 mM, about 250 mM, about 275 mM, about 300 mM, about 325 mM, about 350 mM, about 400 mM, about 450 mM, or about 0.5 M. In a further preferred embodiment, a method is provided wherein the complexing buffer comprises Mg²⁺ in a concentration of about 250 mM.

In order to maintain the slightly basic pH during the processing, and possibly compensating for any pH variations due to the liquid sample, the complexing buffer may further comprise any buffer solution known to the person skilled in the art that maintain a constant pH over a given range by neutralizing the effects of hydrogen ions, e.g. that maintain the pH between 6.95 and 9.1, preferably a pH between 7 and 9, more preferably a pH between 7.2 and 8.8, or between 7.5 and 8.5 or even between 7.7 and 8.3, even more preferably the pH is about 8. Preferred buffers are inert to the further processing of the nucleic acids, such as HEPES, Tris-acetate or Tris-HCl buffers. A preferred buffer is a Tris-HCl buffer, preferably at a concentration of about 0.01 M to 0.1 M, such as 0.02 M to 0.09 M, or 0.03 M to 0.075 M, 0.04 M to 0.06 M, more preferably about 0.05 M. In a preferred embodiment, a method is provided, wherein the complexing buffer comprises QUATS, free cation, and 0.01-0.1 M Tris-HCl, at a pH between 7-9.

Although not necessary for the invention per se, as demonstrated in the examples, addition of butanol has a positive effect on the readout, which was observed uniformly over all different target sequences tested. Hence, addition of butanol can increase robustness of an assay. In an embodiment, the method of the invention is provided, wherein the complexing buffer comprises QUATS, free cation at a pH between 7-9 and butanol at a concentration between 1-10%, such as 2-9%, 3-8% or 4-7%, preferably between 4.5-5.5%, most preferably about 5%.

In another embodiment, the present invention relates to a complexing buffer comprising quaternary ammonium salt (QUATS) and free cation, and has a pH between 7-9, such as between 7.2 and 8.8, or between 7.5 and 8.5 or even between 7.7 and 8.3, even more preferably the pH is about 8, wherein the QUATS is a quaternary ammonium cation having a nitrogen atom at the center and being represented by formula (I)

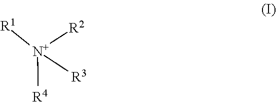

wherein each of R¹, R² and R³ are independently methyl groups (—CH₃) groups, and wherein R⁴ is an alkyl group (CₙH₂ₙ₊₁), with various even-numbered alkyl chain lengths, preferably n=8, 10, 12, 14, 16 or 18, wherein the free cation is chosen from the group consisting of Mg²⁺, Mn²⁺, Li⁺, Nat, Zn²⁺, K⁺ and Ca²⁺, more preferably Mg²⁺, Na⁺ or K⁺ and even more preferably the free cation is Mg²⁺, wherein the concentration of the free cations is between 50 mM and 0.5 M, between 75 mM and 450 mM, between 100 mM and 400 mM, between 150 mM and 350 mM, between 175 mM and 325 mM, between 200 mM and 300 mM, between 225 mM and 275 mM, or about 250 mM.

As mentioned above, the present invention effectively switched the ratio of sample:extraction buffers compared to contemporaneous Boom-protocols, which facilitated analysis of more sample in the fixed dimensions of a cartridge. Most of the present examples used a ratio of 4 ml:3 ml to 4.2 ml:3 ml liquid sample: complexing buffer. However, the present QUATS-process was very flexible in this ratio, in that the amount of the liquid sample could be varied versus the complexing buffer (CB), while still resulting in good purification of NA. The method still gave adequate purification results, in the sense of purity and recovery of NA, using liquid sample: CB buffer ratios ranging from 0.8 to 2.2. In a preferred embodiment, a method according to the invention is provided, wherein the liquid sample is provided in step (i) in a liquid sample: CB ratio between 0.8 and 2.2, preferably between 0.9 and 2.1, more preferably between 1 and 2, such as between 1.1 and 1.8, or between 1.2 and 1.6, more preferably between 1.3 and 1.5. In another preferred embodiment, a method according to the invention is provided, wherein the liquid sample is provided in step (i) in a liquid sample: CB ratio of about 0.8, about, 0.9, about 1, about 1.1, about 1.2, about 1.3, about 1.4, about 1.42, about 1.5, about 1.6, about 1.7, about 1.8, about 1.9, about 2, about 2.1 or about 2.2, most preferably about 1.4 or 1.42.

The methods of the present invention purified the NA to a great extent, in that contaminants were effectively removed. The method also resulted in an abundant recovery of NA, not only compared to the Boom-protocol but also in absolute terms as demonstrated in the examples (cf. for instance the recovery of the spiked targets). In a preferred embodiment, a method according to the invention is provided, wherein the NA in the liquid sample are recovered more than 50%, such as more than 60%, or 70%, or even more than 75%, such as more than 80%, 90% or even more than 95%. In this case, the percentage recovery refers to the amount of NA purified after conducting the method of the invention as a percentage of the total amount of NA present in the liquid sample, before conducting the method of the invention.

The silica support can be prepared using different types of silica materials, including, but not limited to: glass particles, glass powder, silica particles, glass microfibers, diatomaceous earth, silica sand, silica gel, silicon carbide (SiC) and mixtures thereof. Further, the silica support can be prepared using silica materials in various forms. The silica materials can be used in a slurry or bead-based format. In other embodiments, the silica materials may be formed or incorporated into structures, such as silica membranes, silica impregnated or coated filters, and silica coated magnetic beads. Hence, in an embodiment, the present invention provides a method for purifying nucleic acids from a mixture, such as a solution, wherein the silica support is chosen from silica membranes, silica impregnated or coated filters, and silica coated magnetic beads.

As used herein, the term “nucleic acid(s)” (NA) refers to RNA or DNA that is linear or branched; single or double stranded; native, modified or synthesized; or any fragment thereof.

In an embodiment, the nucleic acid is DNA. In a further embodiment, the DNA can be cell free DNA (cfDNA) or circulating tumor DNA (ctDNA). The DNA can be fragmented and/or double stranded.

In the present context, the term “mixture” refers to combination of two or more substances which are mixed in the form of solutions, suspensions or colloids. Preferably, the mixture is a solution, and even more preferably a liquid solution. Unless the context requires otherwise, when referring herein to “solution” a “liquid solution” is intended. A solution is a homogeneous combination composed of two or more substances, in which the solute is a substance, e.g. nucleic acids, dissolved in another substance, known as a solvent. Preferably, the solution comprising nucleic acids is a biological sample comprising nucleic acids.

As used herein, the term “sample” refers to any solution comprising nucleic acids to be purified. The method of the invention is not limited to a specific origin of the sample. The sample may be any of a number of types of samples. In other words, the term “sample” may denote the origin of the NA in solution.

Preferably, the sample is a biological sample.

By “biological sample” is meant a sample comprising biological material, such as a sample comprising cells. For example, a sample comprising cells derived from a cell culture, e.g. from a cell pellet or cell suspension. For example, a sample of body tissue or body fluid, or a biopsy sample. For example, the biological sample comprises bacteria, tissue culture cells, algae, plant stems, roots, or leaves. In some cases, the biological sample may comprise an entire organism.

In a preferred embodiment, the biological sample is a tissue sample. In another preferred embodiment, the nucleic acids are derived or originate from a tissue sample. For instance, the nucleic acids in a tissue sample are released from its nucleoprotein complex or from the cell using a lysis step into a solution comprising the said nucleic acids derived from said tissue.

By “tissue sample” is meant a collection of cells obtained from the same origin of a subject or patient.

The tissue sample may be derived from a solid tissue, i.e. a solid tissue sample, for example, the solid tissue sample is from breast, lung, colon, rectum, brain, heart, liver, spleen, thyroid, thymus, kidney, testis, ovary, intestines, tumors, tissue biopsies; or the tissue sample may be derived from a liquid sample, i.e. a liquid tissue sample, for example, the liquid tissue sample is from blood or any blood constituents, such as white blood cells, serum, plasma, bodily fluids such as bone marrow aspirates, cerebral spinal fluid, amniotic fluid, lung pleural effusion, peritoneal fluid, interstitial fluid, saliva, sweat, tears, breast milk, semen, stool, pleural fluid, or a suspension of cells and other body fluids such as derived from washings, etc. In an embodiment, the biological sample is a solid tissue sample or a liquid tissue sample.

The source of the tissue sample may be a tissue as from a fresh, frozen and/or preserved organ or tissue sample or biopsy or aspirate. The tissue sample may contain compounds which are not naturally intermixed with the tissue in nature such as preservatives, anticoagulants, buffers, fixatives, nutrients, antibiotics, or the like.

In a preferred embodiment, the sample is a biopsy sample.

In still another embodiment the biological sample may be a biological sample of cell specimens, including cell lines, tissues, fine needle aspirates, cell preparations, blood, other biological fluids, bone marrow, cytology specimens, blood smears, thin-layer preparations, imprints, cytospins, cell block sections, micro-arrays, and, frequently, biopsy specimens, disposed on microscope slides, cultured cells or a sample derived from cultured cells, such as cell pellet or culture medium collected from cultured cells.

Blood (plasma, serum or whole blood alike) is the most commonly described fluid used in biopsy sample analysis in humans, which contains a vast array of markers, including exosomal RNA, such as microRNAs (miRNAs) and messenger RNAs (mRNAs), for establishing diagnoses. In cancer patients, blood is the source of circulating tumor cells (CTCs), and cfDNA and cell-free RNA (cfRNA) including ctDNA and circulating tumor RNA (ctRNA), respectively, released by tumor tissues, which can be used to detect mutations present in the patients' tumors. However, ctDNA comprises only a tiny fraction of cfDNA present in the blood, which highlights the importance of maximizing sample volumes for nucleic acid analyses in order to detect rare mutations. Furthermore, cfDNA is often of low quality and fragmented to the approximate size of a nucleosome (140 bp). Consequently, for certain cancer types, including kidney, prostate, and upper and lower tract urothelial carcinomas, alternative biopsy approaches using urine may be a richer source of tumor-derived material. Urine also has other unique benefits such as ease of acquisition (does not require trained medical staff), lack of patient discomfort (increased patient compliance), and may have fewer contaminating proteins compared to blood. Urine, however, still is a very diluted material and consequently its use in diagnostic approaches, especially on LoC devices, would also benefit from maximization of sample input volumes. In view of the currently existing need for NA extraction chemistries that allow maximizing blood or urine sample volume inputs, especially inside of integrated LoC devices like fluidic cartridges, and because present methods are very much suited for this purpose, in another exemplary embodiment, a method is provided wherein the biopsy sample is selected from plasma, serum, whole blood, or urine.

In an embodiment, a method is provided wherein the nucleic acid is DNA, which despite being relatively diluted in biopsy samples, is more stable than RNA, and can be isolated using the disclosed methods at efficiencies similar to or even better than the ones of Boom-extraction-based protocols.

In further embodiments, a NA to be purified from a mixture, such as a solution, is released from its nucleoprotein complex or from a cell using a lysis step, resulting in a lysate. Current methods for lysing are based on mechanical lysis, thermal lysis, chemical lysis, biochemical lysis or electrical lysis, all common in the art. After lysis, the nucleic acid is freed from the nucleoprotein complex or cell. Therefore, in another embodiment, the method of the invention, i.e., QUATS-process, is preceded by a lysis step, for example chosen from mechanical lysis, thermal lysis, chemical lysis, biochemical lysis or electrical lysis. In an embodiment, the present invention relates to a method for purifying nucleic acids from a mixture, such as a solution, comprising nucleic acids, the method comprising the steps of:
(o) performing a lysis on the mixture, such as a solution, comprising nucleic acids;
(i) complexing the nucleic acids in the solution of step (o) to a silica support in the presence of a complexing buffer;
(ii) washing the nucleic acids of step (i) with an alcohol; and
(iii) eluting the nucleic acids of step (ii) with an elution buffer;
by which the nucleic acids are purified; wherein the complexing buffer comprises QUATS and free cation, and has a pH between 7-9.

The Boom-extraction-based protocols comprising chaotropic salts to prevent other biomolecules (e.g. proteins, lipoproteins) from precipitating on the silica solid support, inhibit the activity of nucleases and release DNA from histone proteins. As will be known by those skilled in the art, the same effects can be achieved by introduction of a protease for performing a protein digestion step. This can be advantageous in particularly difficult (e.g. old) samples. Therefore, in another embodiment, the QUATS-process of the invention is preceded by a protease treatment, for example with proteinase K.

In an embodiment, the present invention provides a method for purifying nucleic acids from a mixture, such as a solution, comprising nucleic acids, the method comprising: treating the mixture, such as a solution, comprising nucleic acids, with a protease, such as proteinase K; and
(i) complexing the nucleic acids in the mixture, such as a solution, to a silica support in the presence of complexing buffer;
(ii) washing the nucleic acids of step (i) with an alcohol; and
(iii) eluting the nucleic acids of step (ii) with an elution buffer;
by which the nucleic acids are purified from the mixture, such as the solution; and wherein the complexing buffer comprises QUATS and free cation, and has a pH between 7-9.

In a number of cases, the biological samples are treated with a fixative and are encased in an embedding material, such as FFPE to fix and preserve the tissues. Compared to NA purification from most other sample sources, NA purification from fixed and embedded samples requires the additional step of removal of the embedding material. The skilled artisan is knowledgeable on methods to prepare liquid solutions from such fixated and embedded materials (“liquefaction” or “liquefying”), for instance, such as described in Gilbert et al. (PLOS One 2007, 2(6):e537)), Goezl et al. (BBRC 1985, 130:118-126), WO2012/075133, and particularly WO2014/128129. In general, methods for liquefying FFPE samples include a de-waxing step, which removes the wax, followed by a proteinase K step, liberating the NA from the de-waxed samples, and de-crosslinking the NA at elevated temperatures e.g. at 80-90° C. Commercial kits can be used for extraction of FFPE-DNA, such as DNeasy Blood and Tissue kit from Qiagen, Quick-DNA FFPE Miniprep from Zymo Research (Irvine, CA), NucleoSpin DNA FFPE XS kit from Macherey-Nagel (Hoerdt, France), and Agencourt FormaPure XL DNA from Beckman Coulter (Brea, CA).

Hence, in another embodiment, the sample is liquefied. For instance, the sample is an FFPE sample which is liquefied according to methods common to the skilled artisan.

In an embodiment, the present invention provides a method for purifying nucleic acids from a mixture, such as a solution, comprising NA, the method comprising: liquefying a solid sample, such as an FFPE sample, preferably including a de-waxing step, a proteinase K step, and/or a de-crosslinking step at elevated temperatures, e.g. at 80-90° C., resulting in a mixture, such as a solution, comprising NA; and
(i) complexing the nucleic acids in the mixture, such as a solution, to a silica support in the presence of complexing buffer;
(ii) washing the nucleic acids of step (i) with an alcohol; and
(iii) eluting the nucleic acids of step (ii) with an elution buffer;
by which the nucleic acids are purified from the mixture, such as the solution; and wherein the complexing buffer comprises QUATS and free cation, and has a pH between 7-9.

Following step (i) of complexing the NA in the liquid sample to a silica support in the presence of QUATS and a free cation, at a pH between 7-9, in step (ii), the NA are washed, which can be performed by standard silica washing procedures as known in the art. The washing procedure ensures the removal of residual non-nucleic acid compounds originating from the liquid sample and/or the complexing buffer.

For example, washing of the complexed nucleic acids can be performed in a mode similar to the methodology used in the original Boom protocol, e.g. a concentrated alcohol can be used. Nonetheless, since the method of the present invention uses less material per unit sample and less noxious materials than the Boom protocol, also less washing material and less rigorous washing is required compared to the Boom protocol, resulting in less CoG and improving TAT.

In an embodiment, the QUATS-process according to the invention is provided wherein the alcohol is chosen from methanol, propan-1-ol, isobutanol, sec-butanol, tert-butyl-alcohol, ethanol, butanol and isopropanol, preferably ethanol, butanol and isopropanol, even more preferably ethanol.

In an embodiment, the QUATS-process according to the invention is provided wherein the alcohol is present in concentration of between 70% to 100%, such as 78% to 99%, 80% to 98%, 85% to 97%, such as 90% to 96%.

In an embodiment, the method according to the invention, i.e., QUATS-process, is provided wherein the alcohol is present in concentration of about 70%, about 75%, about 78%, about 80%, about 85%, about 90%, about 92%, about 95%, about 96%, or about 99%, preferably about 96%. In a further preferred embodiment, the method according to the invention is provided wherein the alcohol is ethanol at a concentration of about 96%.

In step (iii) of the QUATS-process according to the invention, the NA are eluted from the silica support, resulting in purified nucleic acids. The elution mechanism can be very similar to that known from contemporaneous methods, wherein the nucleic acids are removed from the silica support into the elution solution. For example, a low ionic strength solution at neutral or slightly basic pH can be used. The present inventors have observed that the pH and the presence or absence of divalent cations in the elution buffer can influence elution efficiency, probably because the charge repulsion between the negatively charged silanol groups and the negatively charged phosphate backbone plays a role during the elution step. The absence of small and/or divalent cations promotes elution efficiency, possibly due to the lack of electrostatic shielding. These mechanisms are generally known in the field, and therefore the choice of a protocol-appropriate wash and elution strategy will constitute no major problem to a skilled person and will not be discussed here further. Nonetheless, the skilled artisan will appreciate that the elution buffer is preferably compatible with or can be used as such in any downstream application, such as, e.g. PCR and NGS.

In an embodiment, the QUATS-process comprises an elution buffer which comprises a low ionic strength solution at neutral or slightly basic pH. In an embodiment a method is provided, wherein the elution buffer has a pH between 8 and 9, such as between 8.1 and 8.7 or more preferably between 8.3 and 8.6, even more preferably wherein the elution buffer has a pH of about 8.4. In an embodiment a method is provided, wherein the elution buffer has a pH of about 8, about 8.1, about 8.3, about 8.4, about 8.6, about 8.7 or about 9. In an embodiment a method is provided, wherein the elution buffer is H₂O or a regular PCR buffer. In a preferred embodiment a method is provided, wherein the elution buffer comprises:
Tris-HCl at a concentration between 0.01 M and 0.05 M, such as between 0.02 M and 0.04 M, preferably about 0.032 M, at a pH 8 to pH 9, preferably a pH of about 8.4;
MgCl₂ at a concentration between 3 mM and 11 mM, such as between 4 mM and 8 mM, preferably about 4.6 mM; and
KCl at a concentration between 0.04 M to 0.15 M, such as between 0.06 M and 0.12 M, preferably about 0.096 M.

As used herein, the terms “purified nucleic acids” or “the nucleic acids are purified”, which are used interchangeably but depending on the structure of the sentence, refers to a solution (i.e. the elution buffer) comprising nucleic acids having fewer contaminants or impurities relative to the solution (liquid sample) prior to the purification method.

The term “purification” or “purify” means that a desired nucleic acid in a sample is separated from undesired components. The term “nucleic acid purification” thus refers to methods for purifying an NA of interest from a solution (the liquid sample) comprising the NA of interest and impurities. Thus, after purification the NA is present in a purer form than before purification. This means that undesired components are present at lower amounts relative to the amount of the desired NA than before purification. As used herein, the term “impurity” or “contaminant” is understood to mean an undesirable component. Undesired constituents of NA-containing samples which may need to be separated from the eventually desired NA may include QUATS, lipids, urea, salts, polysaccharides, proteins, including enzymes such as proteases, RNases and DNases, etc.

The invention provides a method for purifying nucleic acids from a liquid sample, wherein the NA is purified to at least 60% purity, such as at least 70%, at least 75%, at least 80%, at least 85%, at least 90%, at least 95%, at least 97%, such as at least 99% purity, e.g. >99.5%, >99.9%, or even ≥99.95%. In the present context, a percentage purity refers to percentage of nucleic acids compared to the total components (i.e. contaminants or impurities) in the elution buffer.

The purification method of the present invention can be executed in less than 12 hours, such as, e.g. <8 hours, <6 hours, <4 hours, or <2 hours, or even <1 hour, which is used to remove impurities from mixtures, such as solutions comprising a particular NA of interest.

In alternative embodiments of the disclosed methods, a kit and/or a fluidic cartridge are provided comprising any of the above described complexing buffers.

In another embodiment, the method is performed inside of a fluidic cartridge, possibly being a closed fluidic cartridge, likely forming part of an automated system. The cartridge can be of the type that directly accepts a biological sample, obtains a PCR-grade nucleic acid from the sample using the NA purification chemistry of the present invention, and is suitable and adapted to house at least one PCR reaction.

The disclosed methods, kits, kits of parts, systems, or components, relate to a cartridge for an automated system, possibly a LoC-system or -device.

As used herein, the term “cartridge” is to be understood as a self-contained assembly of chambers and/or channels, which is formed as a single object that can be transferred or moved as one fitting inside or outside of a larger instrument that is suitable for accepting or connecting to such cartridge. A cartridge and its instrument can be seen as forming an automated system, further referred to also as an automated platform. In some embodiments, the system further comprises one or more reaction components, such as oligonucleotide mixtures, reporter molecules, and reagents for amplification reactions, such as buffers, salts, enzymes, etc., for instance, a PCR mix. Some parts contained in the cartridge may be firmly connected whereas others may be flexibly connected and movable with respect to other components of the cartridge. Analogously, as used herein the term “fluidic cartridge” shall be understood as a cartridge including at least one chamber or channel suitable for treating, processing, discharging, or analyzing a fluid, preferably a liquid. An example of such cartridge is given in WO2007/004103. Advantageously, a fluidic cartridge can be a microfluidic cartridge. In the context of fluidic cartridges the terms “downstream” and “upstream” can be defined as relating to the direction in which fluids flow in such cartridge. Namely, a section of a fluidic path in a cartridge from which a fluid flows towards a second section in the same cartridge is to be interpreted as positioned upstream of the latter. Analogously, the section to which a fluid arrives later is positioned downstream with respect to a section which said fluid passed earlier. In general, as used herein the terms “fluidic” or sometimes “microfluidic” refers to systems and arrangements dealing with the behavior, control, and manipulation of fluids that are geometrically constrained to a small, typically sub-millimeter-scale in at least one or two dimensions (e.g. width and height or a channel). Such small-volume fluids are moved, mixed, separated or otherwise processed at microscale requiring small size and low energy consumption. Microfluidic systems include structures such as micro-pneumatic systems (pressure sources, liquid pumps, microvalves, etc.) and microfluidic structures for the handling of micro-, nano- and picoliter volumes (microfluidic channels, etc.). Exemplary and very suitable in the present context fluidic systems were described in EP1896180, EP1904234, and EP2419705. In line with the above, the term “chamber” is to be understood as any functionally defined compartment of any geometrical shape within a fluidic or microfluidic assembly, defined by at least one wall and comprising the means necessary for performing the function attributed to this compartment. Along these lines, “amplification chamber” is to be understood as a compartment within a (micro) fluidic assembly, suitable for performing and purposefully provided in said assembly in order to perform amplification of NA. Examples of an amplification chamber include a PCR chamber and a qPCR chamber. In accordance with the above, in alternative embodiments, such cartridges may comprise generic oligonucleotide probes. The terms “chamber” and “compartment”, including the plural versions, are used interchangeably herein, unless the context requires otherwise.

As used herein, the term “automated system” is to refer to integrated platform comprising an instrument and disposable material, such as plastics and solutions, which the system uses in an automated manner to complete a certain process. Such process can be initiated by a user but throughout its automated processing within the system, the user's intervention is not necessary until the process completion. As used herein the term “instrument” is to be understood as a machine equipped with a user interface (e.g. comprising at least a start button or an electricity plug), and/or an onboard computer with software, and programmed to perform certain functions, such as, for instance, running an assay, which can e.g. involve mixing, sonication, heating, data detection and collection, and possibly analysis, etc. In a possible embodiment, the interface can be in a form of a console comprising a computer system running user interface software capable of initiating tests, displaying test results, and communicating with external information systems. An exemplary automated system capable of readily accommodating the present methods is diagnostic platform Idylla™ manufactured by Biocartis NV, which uses a disposable reagent-bearing cartridge that is engageable with a cartridge-processing instrument and provides sample-to-result analytical performance.

As used herein, the term “kit” is to be interpreted as a set of objects comprising at least one article or an assembly or articles or equipment needed for a specific purpose, like performing a molecular biology process or an assay, and preferably a manual. A kit may be provided in a form of a standard benchtop NA purification kit comprising containers with reagents such as the complexing buffer, wash buffers, etc. and e.g. one or more silica supports, spin columns, membranes, beads, or the like. Alternatively, the kit may comprise a cartridge or simply be provided in a form of a cartridge. Along these lines, in a further embodiment, a cartridge is provided, said cartridge comprising a silica support and one or more containers containing the complexing buffer according to the invention, an alcohol and an elution buffer.

In a preferred embodiment, a cartridge is provided configured to purify NA, the cartridge comprising at least 3 containers for storing liquids, at least a sample chamber adapted for receiving a sample, and a processing chamber comprising a silica solid support, wherein containers are in fluid connection with the sample chamber and/or the processing chamber, and wherein.
at least one container containing the complexing buffer according to the invention;
at least one container containing an alcohol; and
at least one container containing an elution buffer;
   wherein the complexing buffer according to the invention comprises QUATS and free cations, and wherein the pH is between 7-9, wherein the QUATS having a nitrogen atom at the center is represented by formula (I) and wherein each of R¹, R² and R³ are independently methyl groups (—CH₃) groups, and wherein R⁴ is an alkyl group (CₙH₂ₙ₊₁), with various even-numbered alkyl chain lengths, preferably n=8, 10, 12, 14, 16 or 18, and wherein the counter-anion is Cl⁻ or Br⁻.

In an embodiment, the cartridge is provided comprising a container with CB, wherein the QUATS of the invention are present in a concentration (mass/vol) between 0.5% to 7%, preferably between 0.75% to 5%, between 0.9% to 4% or between 1% to 3%. In a preferred embodiment, a cartridge is provided wherein the QUATS of the invention are present in a final concentration (mass/vol) of 0.5%, 0.6%, 0.7%, 0.75%, 0.8%, 0.9%, 1.0%, 1.1%, 1.25%, 1.5%, 1.75%, 2%, 2.3%, 2.5%, 3%, 3.5%, 4%, 4.5%, 4.6%, 5%, 5.5%, 6%, 6.5%, 6.9% or 7%. In a further preferred embodiment, a cartridge is provided comprising a container with CB, wherein CTAB is present in a final concentration of 2.3% (mass/vol), DoTAB is present in a final concentration of 1.1% or wherein DoTAC is present in a final concentration of 1.1%.

In an embodiment, a cartridge is provided comprising a container with CB, wherein the free cations of the invention are present in a concentration between 50 mM and 0.5 M, or between 100 mM and 400 mM or between 150 mM and 350 mM, preferably between 175 mM and 325 mM, such as between 200 mM and 300 mM, or 225 mM and 275 mM, or most preferably about 250 mM. In a preferred embodiment, a cartridge is provided wherein the free cations of the invention are present in a final concentration of about 50 mM, about 100 mM, about 150 mM, about 175 mM, about 200 mM, about 225 mM, about 250 mM, about 275 mM, about 300 mM, about 325 mM, about 350 mM, about 400 mM or about 0.5 M. In a further preferred embodiment, a cartridge comprising a container is provided, wherein the free cation is Mg²⁺ in a final concentration of about 250 mM.

In a further preferred embodiment, a cartridge is provided comprising a container, wherein the free cations of the invention are present in a concentration between 50 mM-0.5 M, and wherein the QUATS of the invention are present in a concentration (mass/vol) between 0.5%-7%, at a pH between 7-9.

In a further preferred embodiment, a cartridge is provided comprising at least one container comprising a complexing buffer consisting of Tris-HCl at a pH between 7-9, free cations at a concentration between 50 mM and 0.5 M, and QUATS in a concentration between 0.5% to 7% (vol/vol), and potentially butanol at a concentration between 1-10%.

In a further preferred embodiment, the present invention relates to the use of the method as described herein or of the cartridge as described herein for isolating NA from a mixture, such as a solution.

The presently described NA purification chemistry and related products have the potential of being applied into a wide variety of commercial kits, LoC-devices or disposable cartridges that make use of a solid-phase extraction technology to isolate NA from biological samples. More specifically, its application in fully integrated molecular diagnostic devices could be of great value due to the relatively small volume of the required complexing buffer, which enables increasing of sample input vis-à-vis the necessary buffer volumes. Working examples of the presented concepts are given below.

### EXAMPLES

### Example 1—Experimental Setup

A bench protocol prototype was originally built for which a silica spin column (Macherey-Nagel, blood column nucleospin) was used. In a second phase, a microfluidic prototype was built allowing the automated process from sample input to eluate output. In a third phase, a commercially available standard cartridge was used, in which the original extraction buffer and process were replaced by the CB and QUATS-process of the invention.

The extraction efficiency of NA from plasma was studied based on the following high-level experimental set-up.

For the bench protocol, a silica spin column (Macherey-Nagel, blood column nucleospin) was processed through centrifugation (for a volume up to 1 ml) or was mounted on the QIAvac 24 plus system. This is a vacuum manifold that is connected to a vacuum pump by the QIAvac connecting system. The complete set-up can be used as a flow-through system.

A plasma sample was mixed with proteinase K and subjected to incubation at 37° C. for 10 minutes. Then the complexing buffer or binding buffer was mixed with the lysate and run over the silica spin column. The silica membrane was subsequently washed with ethanol after which the spin column was removed from the collection tube or vacuum manifold.

The spin column was placed in a 1 ml Lo-Bind Eppendorf tube, and subjected to a centrifugation step of 1 minute at full speed to remove traces of ethanol. The spin column was subsequently transferred to a new 1 ml Lo-Bind Eppendorf tube, followed by rehydration of the membrane with elution buffer. After a 2-minute incubation at room temperature (RT), the spin column was subjected to an additional centrifugation step of one minute at full speed.

The eluted product was analyzed by qPCR (quantitative or real-time PCR), providing a relative quantification of the purified nucleic acids.

In the microfluidic device the protocol follows a similar course: plasma was loaded in the cartridge and the volume was adjusted up to 4 ml with Tris-HCl EDTA with 0.1% recombinant albumin, if necessary. Proteinase K was added inside the lysis chamber and homogenized and incubated at low temperature using High Intensity Focused Ultrasound (HIFU). Complexing buffer or binding buffer was added to the lysate, and further homogenized via HIFU, before being sent over a silica membrane where the NA was purified. A washing step was performed by passing highly concentrated ethanol over the silica membrane. Ethanol evaporation was carried over by warming the extraction membrane and passing a large volume of air over it. Elution was performed by passing warmed elution buffer at low speed over the membrane and sending it to the mixing chamber where it can be retrieved for downstream application in external appliance(s). In the fully automated process, the eluate was transferred to the PCR disk where qPCR was performed.

### Example 1.1—Sample Type and Complexing Buffer Chemistry

Pooled or single individual plasma were spiked with a mixture of synthetic target (double-stranded DNA (“dsDNA”) of 100 bp to 300 bp), gDNA (genomic DNA) or fragmented DNA. Spiking provided robust downstream qPCR-based target detection, since plasma from patients is very scarce and valuable, while plasma originating from healthy donors is not expected to contain oncogenic alterations.

One to 4 ml of plasma were spiked with 30 to 2300 copies of synthetic target in a background of 1000 to 60 000 copies of wild type background, except for KIF11, which is naturally present in the samples. Plasma was lysed via addition of 50 μl to 200 μl of proteinase K (for 1 ml to 4 ml of plasma respectively). The lysate was then mixed with the complexing buffer (CB) in a ratio 3:4.2 (CB: lysate).

Unless indicated otherwise, the CB was composed of either 2.3% (m/v) of cetyl-trimethylammonium bromide (CTAB) or 1.1% (m/v) dodecyl-trimethyl ammonium chloride (DoTAC) dissolved in a 0.05 M Tris-HCl pH 8 buffer in the presence of 250 mM of MgCl₂ and 5% butanol. This resulted in a final concentration of 0.96% CTAB or 0.46% DoTAC, 0.021 M Tris-HCl, 104 mM MgCl₂ and 2.1% butanol when mixed with the lysate. This mixture with a total volume of 2.1 ml to 7.2 ml was then run over the silica membrane.

### Example 1.2—Washing Buffer Chemistry and Processing

Washing of the silica membrane was performed by running 900 μl of 78-96% ethanol over the spin column. Subsequently, any residual ethanol traces were removed by subjecting the spin columns to a centrifugation step of one minute at full speed (bench protocol) or by passing 20 ml of air over the membrane (automated prototype).

In an early version of the washing protocol, the ethanol step was preceded by a wash step with a low pH acetate buffer. However, this step was proven to be unnecessary and therefore omitted in later protocols.

### Example 1.3—Elution Buffer Chemistry and Processing

Elution of NA was performed by rehydrating the silica membrane with water or a 32 mM Tris-HCl pH 8.4 buffer containing 4.6 mM of MgCl₂, 96 mM of KCl and 5% proclin. For the bench protocol the elution buffer was warmed to RT, and was contacted with the silica membrane for at least 2 minutes. Subsequently, the spin column was subjected to a final centrifugation step (1 minute, full speed). The eluted product was then recuperated in a 1 ml Lo-Bind Eppendorf tube. For the automated protocol, elution buffer was pumped over the heated extraction membrane at a speed of 2 μl/s and was directed to the mixing chamber of the cartridge.

### Example 1.4—qPCR Design and Conditions

In order to evaluate the extraction efficiency, a pentaplex design was used that is composed of five target amplicons, each with a different size. These target amplicons were located on different genes.

Differences in Cq (Cq=quantification cycle, which is used interchangeably with Ct=cycle threshold) values between the reading and a quantified positive control indicate the extraction yield of the protocol.

For the bench qPCR, 23 μL of the eluted product was mixed with 2 μl PCR mix. The components in the final PCR reaction were 96 mM KCl, 10 mM Tris-HCl pH 8.4, 4.6 mM MgCl₂, 0.2 mM dNTP mix, 300 nM of each primer and probe and 5 units of GoTaq DNA polymerase. The qPCR reaction was performed on a Biorad CFX96 Touch™ Real-Time PCR Detection System. The total reaction volume was 25 μL. The cycling protocol included a hotstart (1 minute, 95° C.) followed by 50 cycles of denaturation (3 seconds, 95° C.) and annealing/extension (10 seconds, 64° C.). The fluorescent signal was measured after each cycle.

For the PCR performed in the automated Idylla™ platform, each PCR chamber was filled with 27 μl of the eluate which dissolved the spot containing the PCR mix. The cycling protocol included a hotstart (50 seconds, 95° C.) followed by 50 cycles of denaturation (5 seconds, 95° C.), annealing/extension (10 seconds, 64° C.). The fluorescent signal was measured after each cycle.

### Example 2—QUATS-Process Outperforms the Gold-Standard GU-Extraction Protocol

The experimental setup as described in Example 1 was used to test the new protocol.

### Example 2.1—Comparison Between CTAB and Classic GU-Based Extraction Protocols

A comparison was made between the QUATS-process as described in Example 1 using CTAB and the classic Guanidinium-based extraction protocol (GU). The GU-based extraction protocol entailed adding binding buffer (BB) consisting of 3.68 M GuSCN, 43% butanol to the lysate in a 1.2:6 ratio (lysate:BB). The final concentration was 3.05 M GuSCN, 25.8% butanol.

Both protocols were tested according to the pentaplex design of Example 1.4. The target sequences detected were KRAS G12V, EGFR C797S, EGFR L858R, HER2 and KIF11. For each condition plasma samples of 4 mL for CTAB (20 samples) and 1 mL for the GU-based extraction protocol (12 samples) were used. The resulting eluates were retrieved from the cartridge and processed in replicates by qPCR.

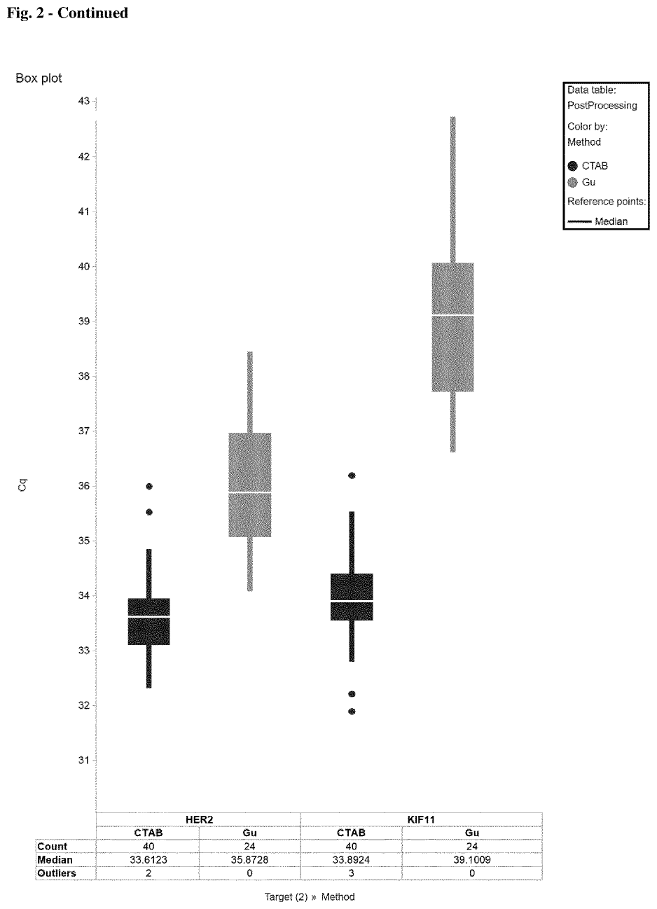

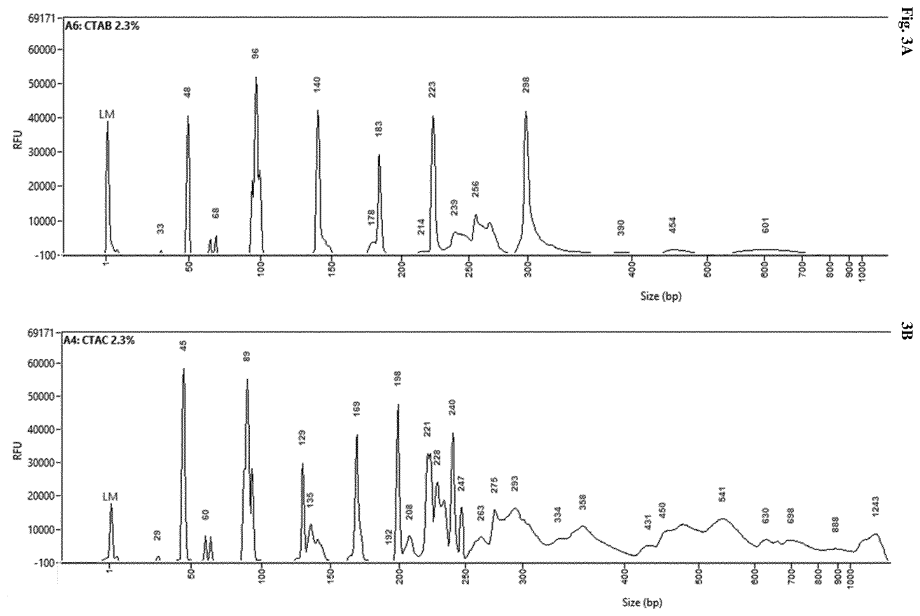

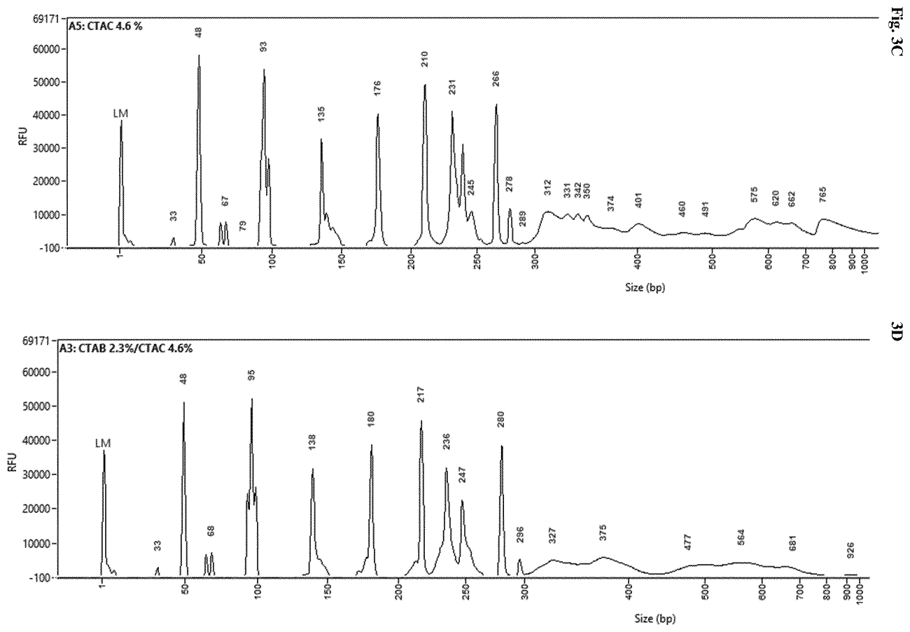

The results are depicted in FIG. 2.

The first observation is that the QUATS-process consistently and efficiently enabled purification from a range of target nucleic acids.

The second observation is that the QUATS-process was at least as efficient as, if not better than the classic GU-based extraction protocol. The ΔCq between the two protocols indicates extraction efficiency differences, i.e. a Δ of 1 Cq=2-fold extraction yield difference. It can be seen that the QUATS-process was consistently more efficient than the classic GU-based extraction protocol, with a ΔCq ranging between 1.5-5.3 (which represents a 2.8 to 39.4-fold increased detection). Due to the differences in volume between the two extraction processes (4 ml for CTAB and 1 mL for GU-based extraction protocol), 4 times more target material is available in the QUATS samples than the GU samples. Hence, there is a difference of about 2 Cq in favour of the QUATS-process.

The third observation is that the QUATS-process required less, less costly and less noxious purification materials than the classic GU-based extraction protocol. These QUATS-process purification materials were also compatible with the cartridge covering.

The fourth observation is that the sensitivity of the QUATS-process compared to the classic GU-based extraction process was increased by a factor 4 in the fixed dimensions of a cartridge.

### Example 2.2—Quaternary Salt Counter-Anion has Minimal Influence

As demonstrated in Example 2.1, the QUATS-process according to the invention performed substantially better than the classic GU-based extraction protocol. Example 2.1 was performed with the exemplary QUATS compound cetyl-trimethyl-ammonium bromide (hexadecyl-trimethylammonium bromide, CTAB, n=16), which gave consistent results in a large set of experiments (not all shown).

In order to examine the effects of the quaternary salt counter-anion, a direct comparison was made between the QUATS-process using CTAB, having a bromide counter-anion, and cetyl-trimethyl-ammonium chloride (CTAC, n=16), having a chloride counter-anion.

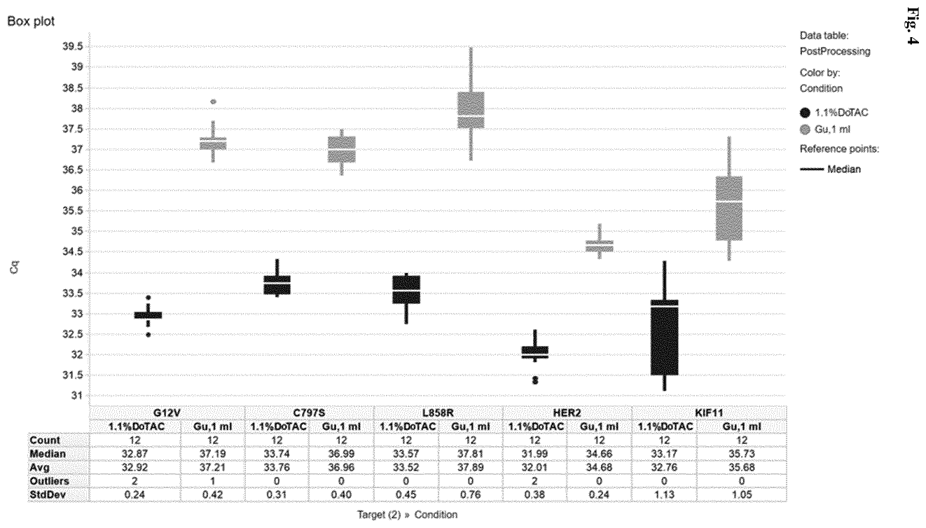

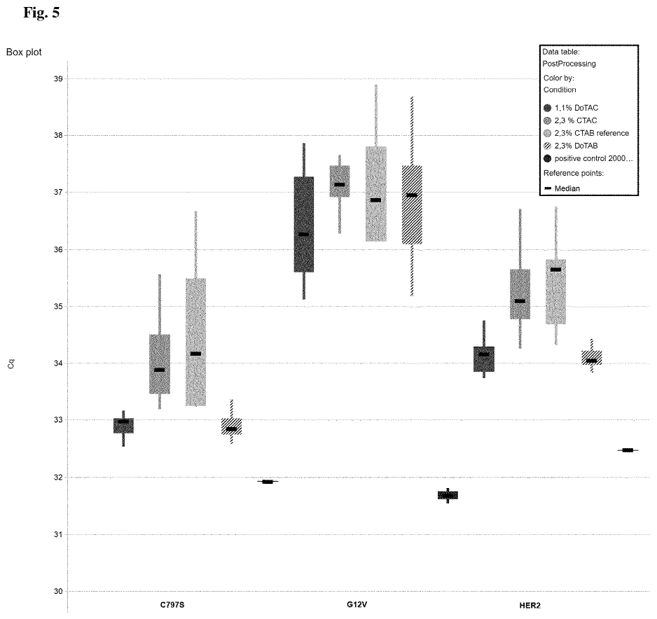

In addition, the effect of various concentrations of QUATS were tested, i.e. 2.3% CTAB (FIG. 3A), 2.3% CTAC (FIG. 3B), 4.6% CTAC (FIG. 3C) and 2.3% CTAB/4.6% CTAC (FIG. 3D).

Complexing buffers were prepared according to Example 1.1. Different concentrations of CTAB and CTAC were dissolved in a 0.05 M Tris-HCl pH 8 buffer in the presence of 250 mM of MgCl₂ and 5% butanol. A DNA ladder was spiked in a single plasma sample prior to extraction. Extraction was carried out with the same sample but with different complexing buffers on the bench, using Macherey Nagel columns according to Example 1. Analysis of the results was performed by fragment analyzer (gel electrophoresis).

The results are shown in FIG. 3.

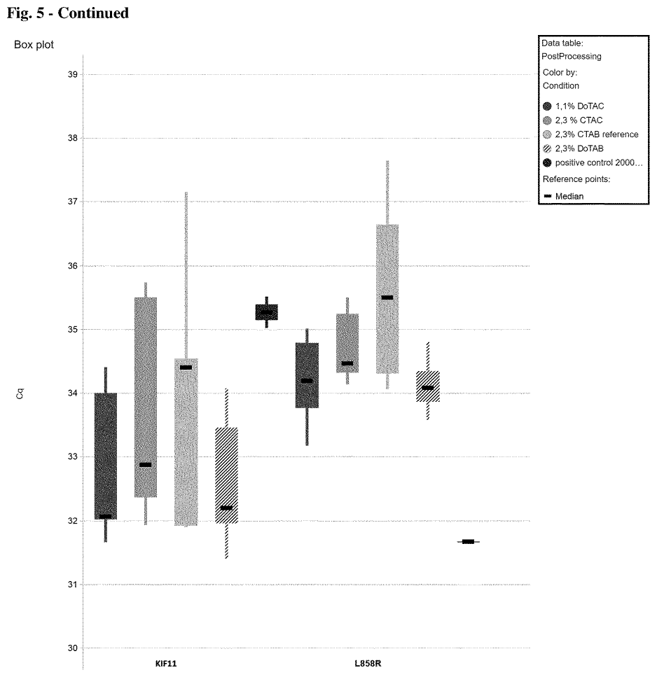

Both complexing buffers comprising either CTAB or CTAC purified the spiked-in DNA ladder adequately, although there seemed to be a small difference in the spectrum of extraction efficiencies (cf. area under the curve), in that QUATS with a bromide counter-anion extracted smaller NA fragments slightly more efficiently than QUATS with a chloride counter-anion, while the latter extracted larger NA fragments slightly more efficiently than QUATs with a bromide counter-anion (right side of the electropherogram; cf. FIGS. 3A & 3B). Thus, the purification protocols according to the invention with QUATS having either a chloride counter-anion or a bromide counter-anion resulted in purified nucleic acids useful for further analysis, especially including analysis via PCR (cf. FIG. 5).

As can be seen in FIGS. 3B, 3C & 3D, the QUATS according to the invention extracted the NA efficiently over a wide range of concentrations, i.e. from 2.3% to 6.9%. In a direct comparison, CTAC at 4.6% seemed to be marginally better than CTAC at 2.3% (cf. FIGS. 3B & 3C).

Since the extraction efficiency spectra seemed to differ slightly between the counter-anions, the inventors hypothesized that a combination of both QUATS would possibly level the differences. FIG. 3D shows a combination of 2.3% CTAC/4.6% CTAB. It appeared that the combination of both counter-anions resulted in an additive effect, in that both smaller and larger fragments were extracted effectively.

In conclusion, all the quaternary salt counter-anions performed successfully. Nonetheless, the bromide counter-anion appeared to extract smaller NA fragments slightly more efficiently than the chloride counter-anion, while the latter extracted larger NA fragments slightly more efficiently than the bromide counter-anion in the QUATS-process of the invention. Moreover, the QUATS could be used in a wide range of concentrations in the QUATS-process of the invention.

### Example 2.3—QUATS-Process Performs Better than Classic GU-Based Extraction Protocols

After demonstrating in Example 2.1 that the QUATS-process according to the invention performed substantially better than the classic GU-based extraction protocol, and demonstrating in Example 2.2 that the counter-anion of the quaternary ammonium salt has no major influence on the purification efficiency, the effect of the length of the aliphatic chain was tested. Indeed, the observations in Examples 2.1 & 2.2 were consistent with the inventors' hypothesis and were predictive of the ubiquitous usefulness of the QUATS-process.

Nonetheless, in order to further substantiate the hypothesis and generalize the observations with the improved performance of the QUATS-process over the classic GU-based extraction protocol, the experiment of Example 2.1 was repeated in essence but now with Dodecyltrimethylammonium Chloride (DoTAC). The essential difference is that DoTAC has 4 methyl-groups less (n=12) than CTAB (n=16). Although DoTAC has chloride as a counter-anion, while CTAB has a bromide as corresponding counter-anion, this would have no actual impact, as demonstrated in Example 2.2. In fact, this was further corroborated in the present example.

The protocols were tested according to the pentaplex design of Example 1.4. The genes detected were KRAS G12V, EGFR C797S, HER2, KIF11 and EGFR L858R. For each condition, 12 plasma samples of 4 ml for DoTAC and 1 ml for GU-based extraction protocol were used. The resulting eluates were retrieved from the cartridge and processed in replicates by qPCR.

The results of the experiment are depicted in FIG. 4. These results confirmed and strengthened the observations of Example 2.1 & 2.2.

Indeed, it was again observed that the QUATS-process consistently and efficiently enabled purification from a range of target nucleic acids. In this case, the QUATS-process was definitely more efficient than the classic GU-based extraction process.

In addition, it could be concluded that the QUATS-process was widely applicable with the different QUATS compounds according to the invention, i.e. QUATS having aliphatic tails with different lengths and QUATS having different counter-anions.

### Example 2.4—QUATS-Process Performance with a Range of QUATS Compounds

The individual teachings of Examples 2.1, 2.2 & 2.3 indicated that the QUATS-process according to the invention performed substantially better with a wide range of QUATS compounds and concentrations than the classic GU-based extraction protocol.

These individual teachings of different QUATS compounds were combined. In particular, complexing buffers were prepared according to Example 1.1. Different QUATS compounds, i.e. DoTAB (at 2.3%), CTAB (at 2.3%), CTAC (at 2.3%) and DoTAC (at 1.1%) were dissolved in a 0.05 M Tris-HCl pH 8 buffer in the presence of 250 mM of MgCl₂ and 5% butanol.

The protocols were tested according to the pentaplex design of Example 1.4. A subset of healthy donor samples was used, in which the particular target sequences were spiked. The alterations detected were EGFR C797S, KRAS G12V, HER2, KIF11 and EGFR L858R. For each condition, 5 plasma samples were used. The resulting eluates were retrieved from the cartridge and processed in replicates by qPCR.

The results are shown in the box plot of FIG. 5 and summarized in Table 1.

TABLE 1

| | | 1.1% DoTAC | 2.3% CTAC | 2.3% CTAB | 2.3% DoTAB | pos control |
|---|---|---|---|---|---|---|
| C797S | Count | 10 | 10 | 10 | 10 | 2 |
| | Median | 32.9785 | 33.8934 | 34.1745 | 32.8473 | 31.9235 |
| | Outliers | 0 | 0 | 2 | 0 | 0 |
| G12V | Count | 10 | 10 | 10 | 10 | 2 |
| | Median | 36.2691 | 37.135 | 36.8693 | 36.9522 | 31.6772 |
| | Outliers | 0 | 0 | 2 | 0 | 0 |
| HER2 | Count | 10 | 10 | 9 | 10 | 2 |
| | Median | 34.1618 | 35.0917 | 35.6544 | 34.0491 | 32.4699 |
| | Outliers | 0 | 0 | 1 | 0 | 0 |
| KIF11 | Count | 10 | 10 | 10 | 10 | 2 |
| | Median | 32.0672 | 32.8742 | 34.4083 | 32.2014 | 35.2726 |
| | Outliers | 0 | 0 | 2 | 0 | 0 |
| L858R | Count | 10 | 10 | 9 | 10 | 2 |
| | Median | 34.2032 | 34.4698 | 35.5072 | 34.0811 | 31.6678 |
| | Outliers | 0 | 0 | 1 | 0 | 0 |

First, it can be seen that for all conditions tested the results were comparable, in that there was only a minor variation between the different QUATS compounds. The main variation appeared to relate to the particular target sequence (cf. Example 2.3).

Next, the results demonstrated that the counter-anions have no influence on the extraction performance and analysis (cf. Example 2.2).

Also, the present experiment confirmed that the QUATS-process is useful with a wide range of concentrations of QUATS compounds, and further substantiated the hypothesis experimentally from 1.1% to 6.9% (cf. DoTAC and Example 2.2).

This low concentration of QUATS compounds still being effective, further decreased costs. Moreover, a low concentration has the additional, potential advantages of (a) less spurious binding of contaminants, and (b) the need to remove less compounds in the subsequent steps of the purification method.

### Example 2.5—Comparative Example—QUATS

As demonstrated in Examples 2.1 to 2.4, using different QUATS compounds (of formula (I)) having one alkyl chain, wherein each of R¹, R² and R³ are independently methyl groups (—CH₃) groups, and wherein R⁴ is an alkyl group CₙH₂ₙ₊₁, the QUATS-process according to the invention performed exceptionally good.

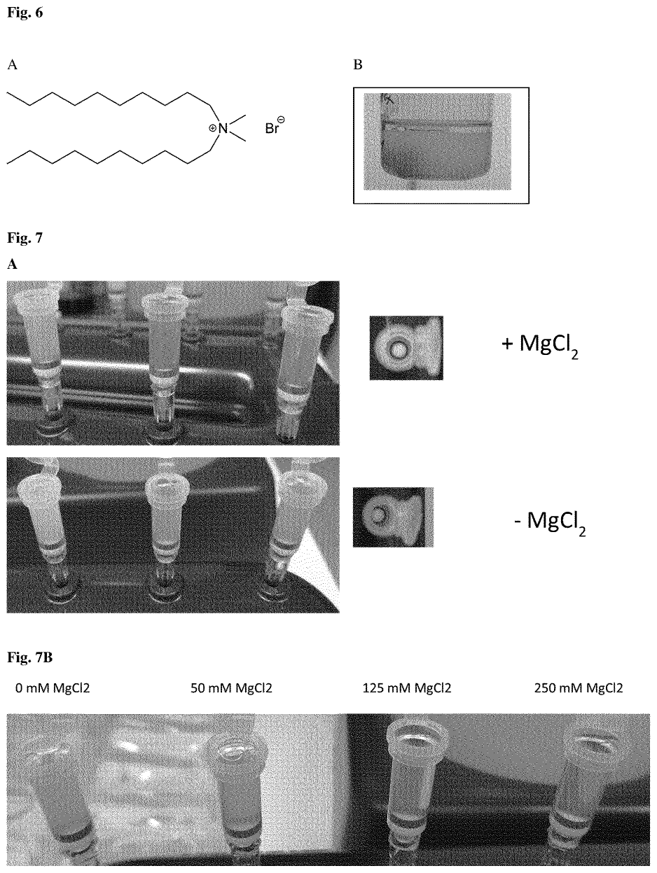

It the present example, the inventors compared the QUATS compounds according to the invention as depicted in FIG. 1 with quaternary ammonium salts having a di-alkyl chain, i.e, wherein R³ and R⁴ are an alkyl group CH₂H₂ₙ₊₁ with n>1. As an exemplary di-alkyl chain, didodecyldimethylammonium bromide (DDAB) was used (FIG. 6A).

A complexing buffer using DDAB was prepared according to Example 1.1.

However, no direct comparison between the effects of a quaternary ammonium salt with two aliphatic chains (di-alkyl chains) and the mono-alkyl chains according to the invention was possible. When adding the di-alkyl chain DDAB to the 50 mM TRIS-HCl buffer pH 8, 250 mM of MgCl₂ and 5% butanol, a phase separation was immediately visible (FIG. 6B), indicating a solubility issue.

Hence, the di-alkyl chains appear not applicable in the QUATS-process of the present invention.

### Example 2.6—Summary Conclusions

The present set of experiments demonstrated that the QUATS-process of the invention is superior to the current golden standard “Boom”-protocol, not only in efficiency, but also in the ratio input-material versus extraction-materials and the safety of the materials. In addition, it was shown that a wide range of mono-alkyl QUATS compounds were useful in this protocol, having different lengths of the alkyl chain, having different counter-anions, and in a broad concentration range varying between 1.1%-6.9% (corresponding to a final concentration of about 0.5%-3.1%).

### Example 3—the Effect of Free Cations & Butanol

### Example 3.1—Free Cations Increase Solubility

The inventors hypothesized that the presence of free cations may act as a bridge between the silica support and the nucleic acids, which may augment complexing the nucleic acids to the silica support. Magnesium (Mg⁺⁺) was used as an exemplary representative of free cations. Notably, magnesium is also part of PCR buffers.

Two different complexing buffer compositions were made and tested, one with and one without Mg²⁺. The free cations, in this case 250 mM Mg²⁺, were added to the CB consisting further of DoTAC (at 1.1%) dissolved in a 0.05 M Tris-HCl pH 8 buffer and 5% butanol. A unique plasma was digested by proteinase K before addition of the CB with or without Mg²⁺. The samples were mixed and loaded on Macherey-Nagel columns according to Example 1.

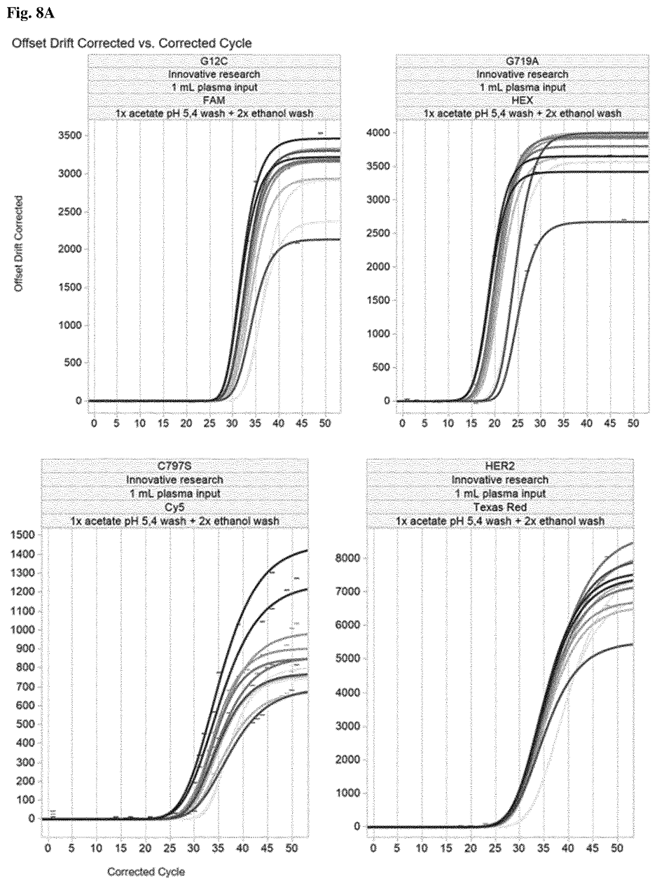

The results are depicted in FIG. 7A.

Already by visual inspection only, it was obvious that the addition of the free cations improved the solubility of the sample. In addition, the flowthrough speed of the columns was also increased, which is another parameter of solubility (not shown), and could help to prevent clogging of the silica support.

In order to determine whether the concentration of the free cation would be of influence on the solubility of the sample, the above experiment was repeated with different concentrations Mg²⁺: 0 mM, 50 mM, 125 mM and 250 mM MgCl₂.

The results are shown in FIG. 7B.

This experiment demonstrated that the addition of 50 mM free cations already facilitated solubility and thus facilitated extraction of the sample. Solubility further improved with increasing the concentration of Mg²⁺ to 250 mM.

### Example 3.2—Free Cations Increase Extraction Yield and Facilitate Analysis

Example 3.1 demonstrated that the addition of free cations increased the solubility of the sample and facilitated flowthrough of the sample. It would thus be expected that extraction yield would also be promoted, which would in turn increase the resolution power of the assay.

The inventors thus set out to determine whether the addition of free cations indeed increased extraction efficiency by detecting and measuring the Cq of various target sequences. Although the Cq was used as a proxy for extraction efficiency, the Cq is in fact a true determinant for the robustness of the eventual assay since it can also be influenced by impurities after extraction.

Five different CB compositions were made and tested, using the exemplary free cation Mg²⁺, i.e. four CB compositions with and one CB composition without Mg²⁺ (negative control). The free cations, in this case 100 mM, 200 mM, 300 mM and 400 mM MgCl₂, were added to the CB consisting further of CTAB (at 2.3%) dissolved in a 0.05 m Tris-HCl pH 8 buffer and 5% butanol.

The different concentrations of free cations were tested according to the pentaplex design of Example 1.4. 2000 copies of the target sequences EGFR C797S, KRAS G12C, EGFR G719A, and HER2 were spiked in 1 ml plasma sample. The samples were processed according to standard conditions and subjected to qPCR.

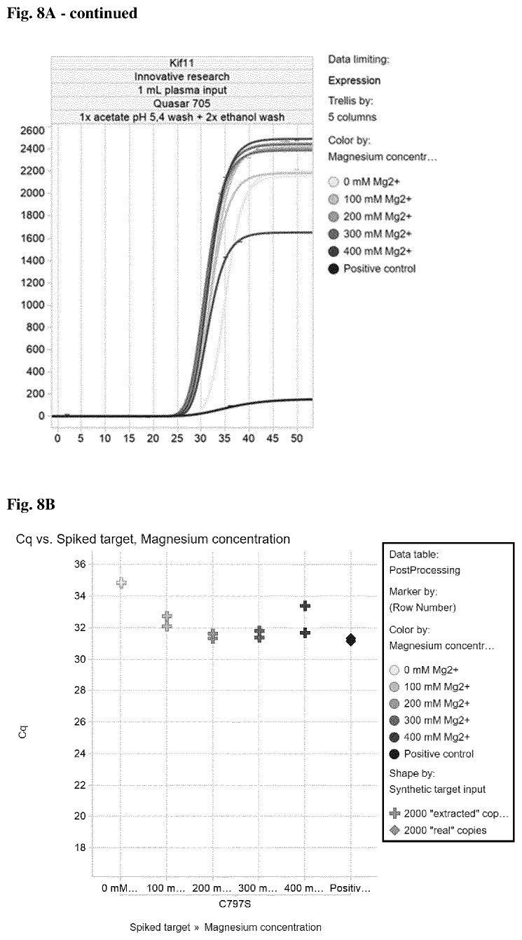

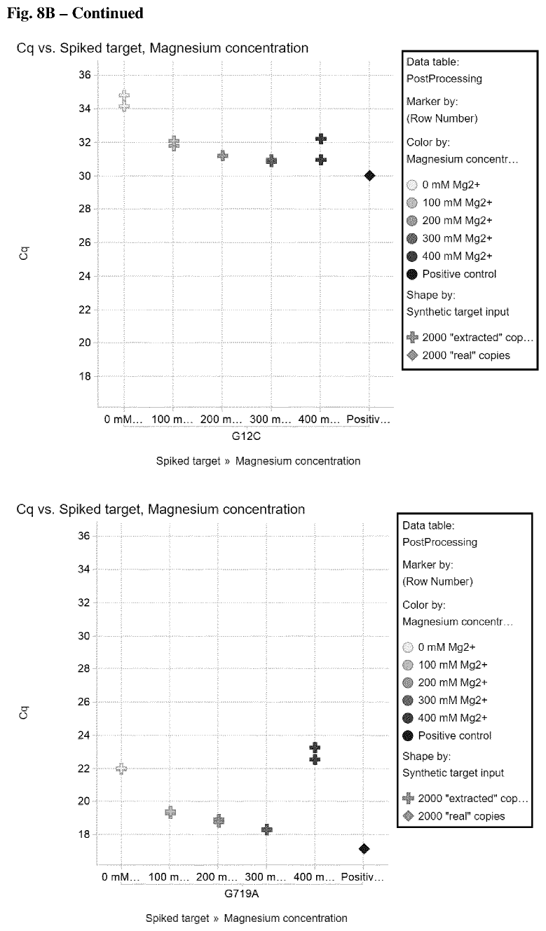

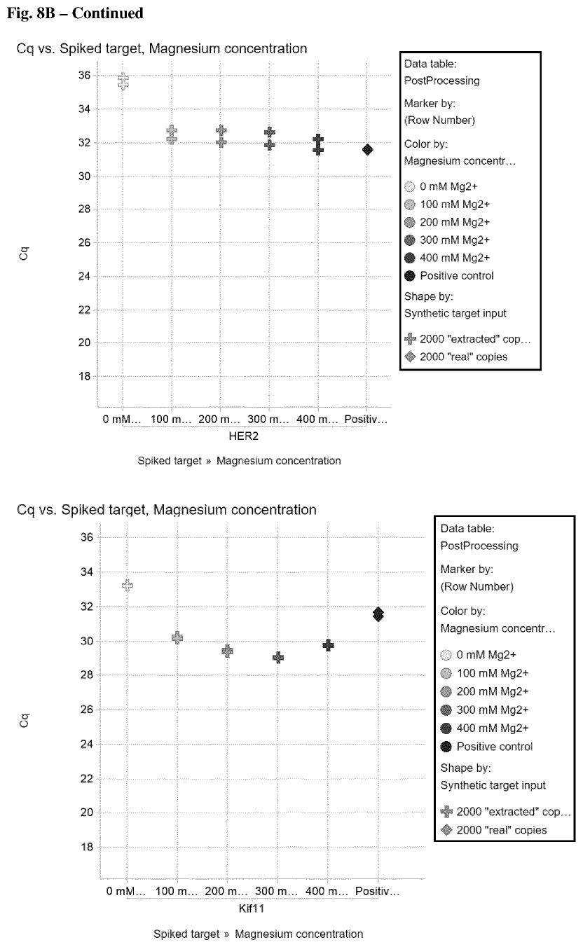

The results are summarized in FIGS. 8A and 8B.

It can be seen that the addition of free cations consistently increased the detection efficiency compared to the samples without free cations. In this example the concentrations ranging from 100 mM to 400 mM all performed well, with no concentration being consistently better or worse over this range.

The above experiment was essentially repeated, now using 1.1% DoTAC and 2000 copies of target sequences spiked into 4 ml plasma sample in an actual standard cartridge (Idylla™) with different concentrations Mg²⁺: 0 mM, 50 mM, 125 mM and 250 mM MgCl₂. The different concentrations of free cations were tested according to the pentaplex design of Example 1.4, using the target sequences EGFR C797S, KRAS G12V, HER2, KIF11 and EGFR L858R. The samples were processed according to standard conditions and subjected to qPCR.

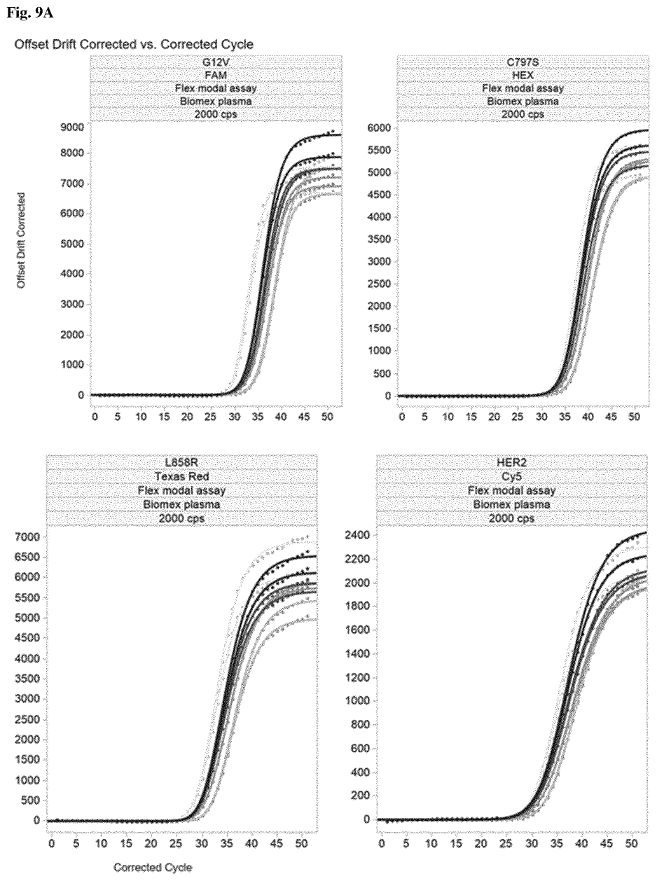

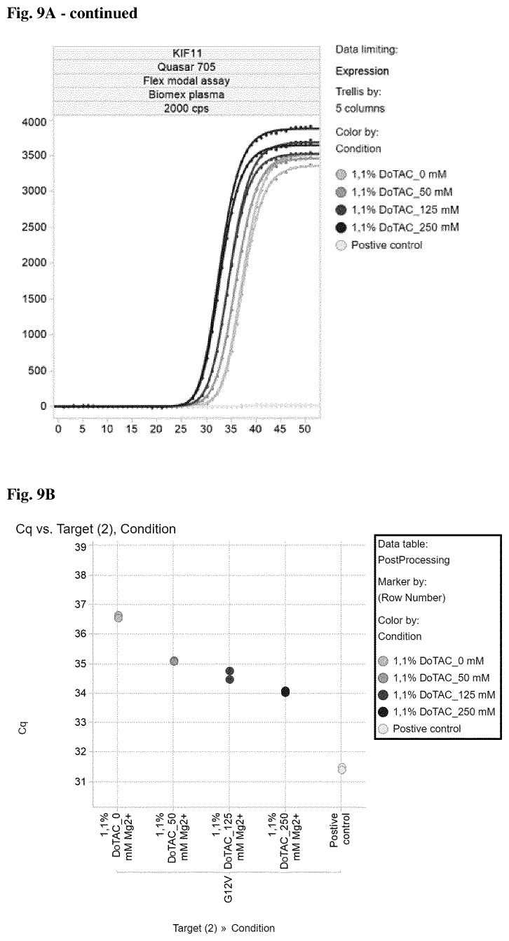

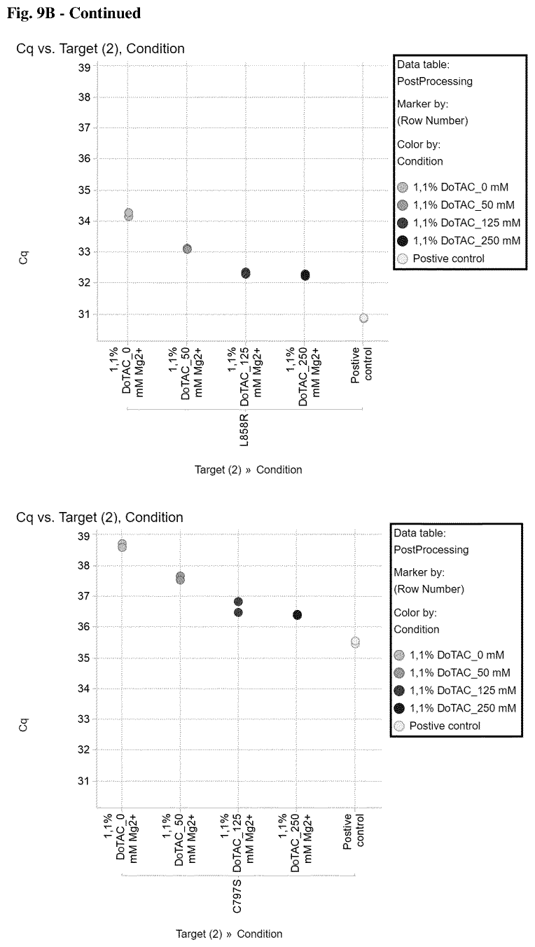

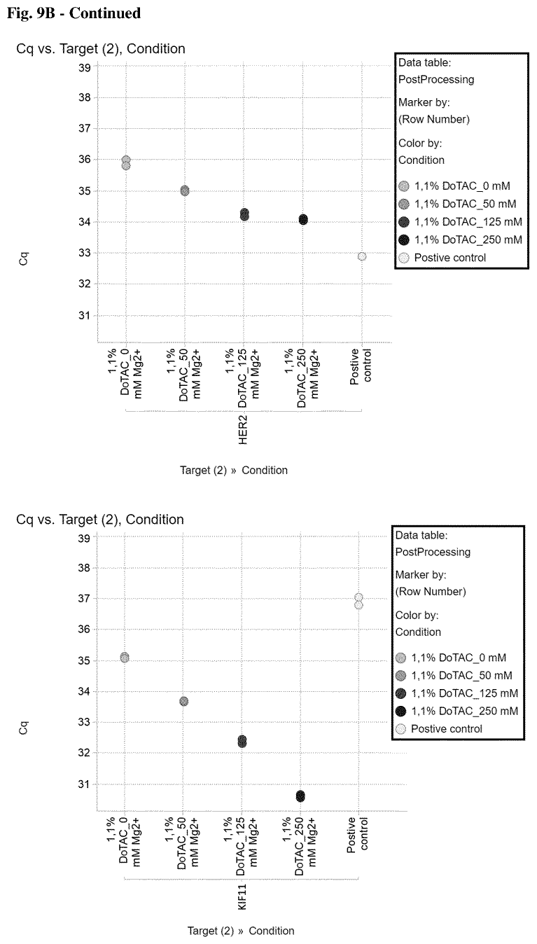

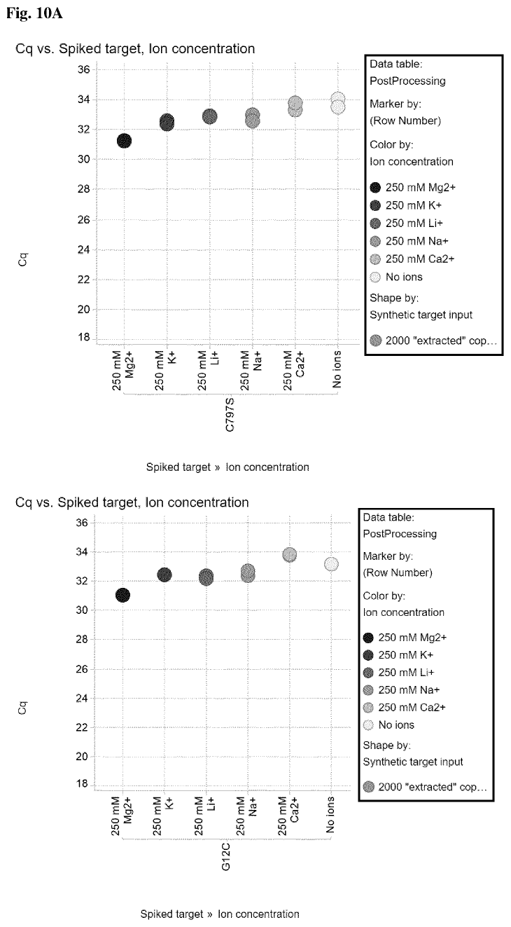

The results are summarized in FIGS. 9A and 9B.

Again, it can be seen that the addition of free cations consistently increased the detection efficiency compared to the samples without free cations. Although the differences between the various concentrations were minute, in this example the concentration of about 250 mM appeared to be the best performer overall.

### Example 3.3—Free Monovalent and Bivalent Cations Facilitate Analysis

A comparison was made between different free cations on the QUATS-process according to the invention.

The protocols were tested according to the pentaplex design of Example 1.4. The NA detected were KRAS G12C, EGFRC797S, EGFR G719A, HER2 and KIF11.

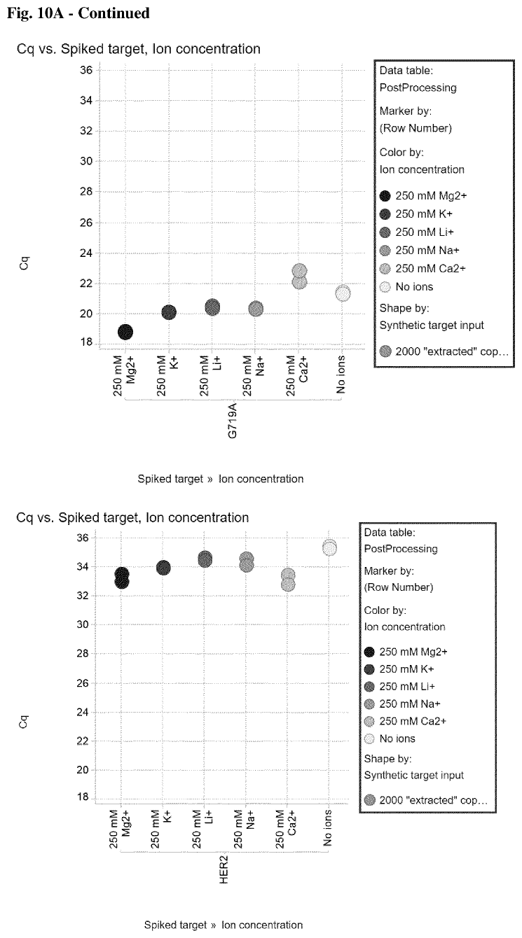

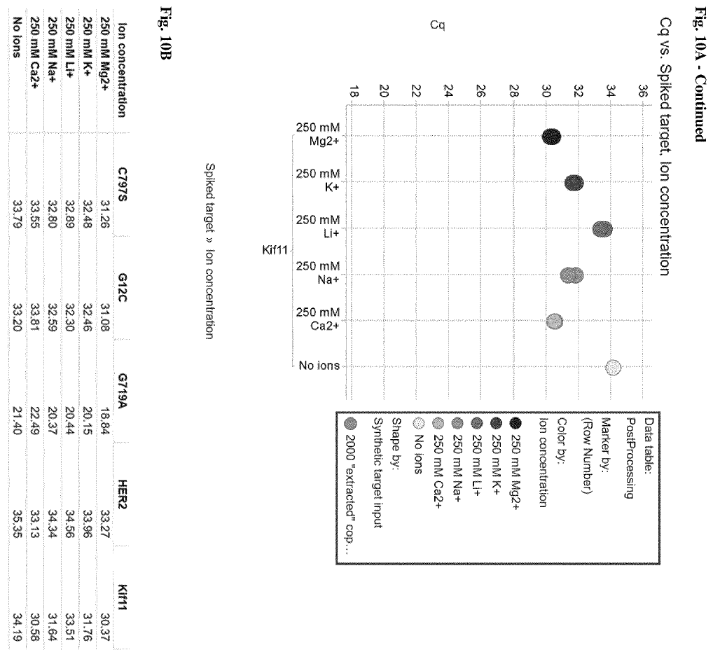

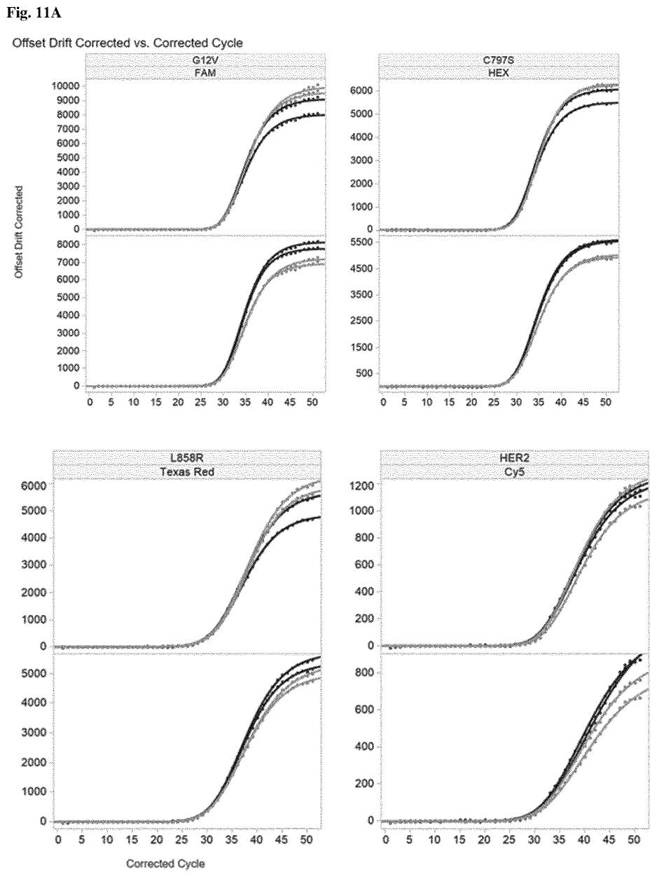

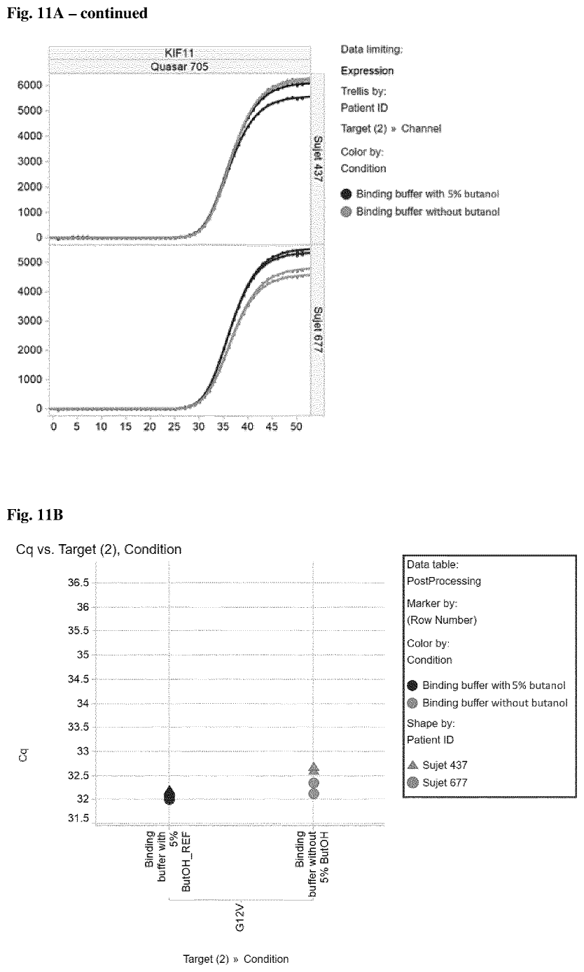

Different CB compositions were tested: a complexing buffer solely containing QUATS, i.e. DoTAC (at 1.1%) dissolved in a 0.05 M Tris-HCl pH 8 buffer in the presence of 5% butanol (negative control), while other complexing buffers were supplemented with free cations. Five different free cations were tested: Mg²⁺, K⁺, Li⁺, Nat, and Ca²⁺, all at a concentration of 250 mM. The cations were added as Cl-salts. The results are summarized in FIGS. 10A and 10B.

This experiment demonstrated that the addition of both monovalent and bivalent free cations mediated the complexing of NA to the silica membrane, and thus facilitated the purification process.

Although all cations tested enhanced the purification process compared to the CB without free cations, Mg²⁺ performed consistently better. Magnesium improved the extraction process by an average of four fold (ΔCq=2).

### Example 3.4—Effect of Butanol in the Complexing Buffer

The inventors studied the effect of butanol in the complexing buffer on the assay robustness.

The protocols were tested according to the pentaplex design of Example 1.4. The sequences detected were EGFR G797S, KRAS G12V, HER2, KIF11 and EGFR L858R. 2300 copies were spiked into a sample. Two different CB buffer compositions were tested: with 5% butanol and without butanol. The samples were processed according to standard conditions and subjected to qPCR.

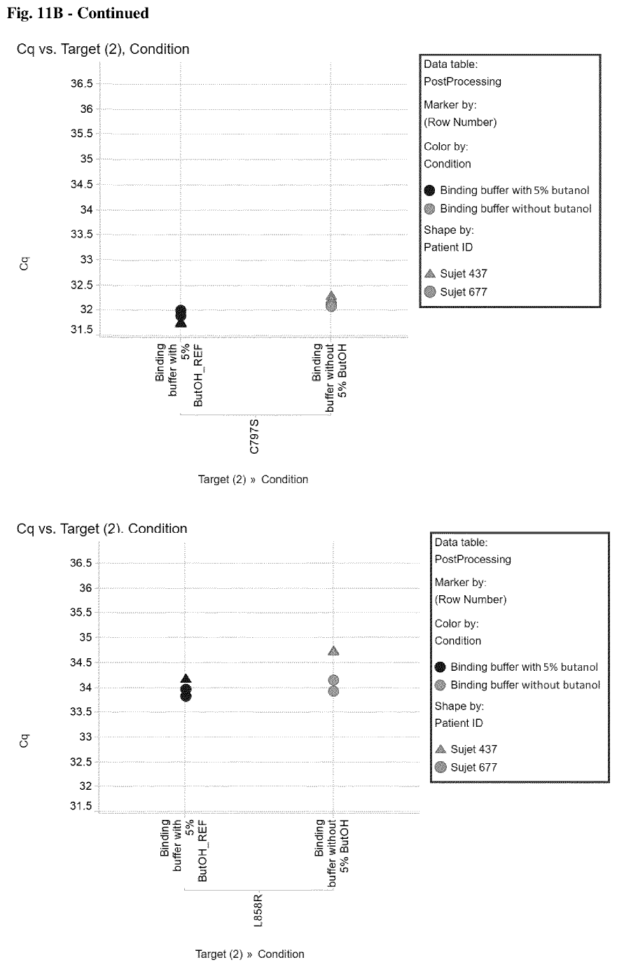

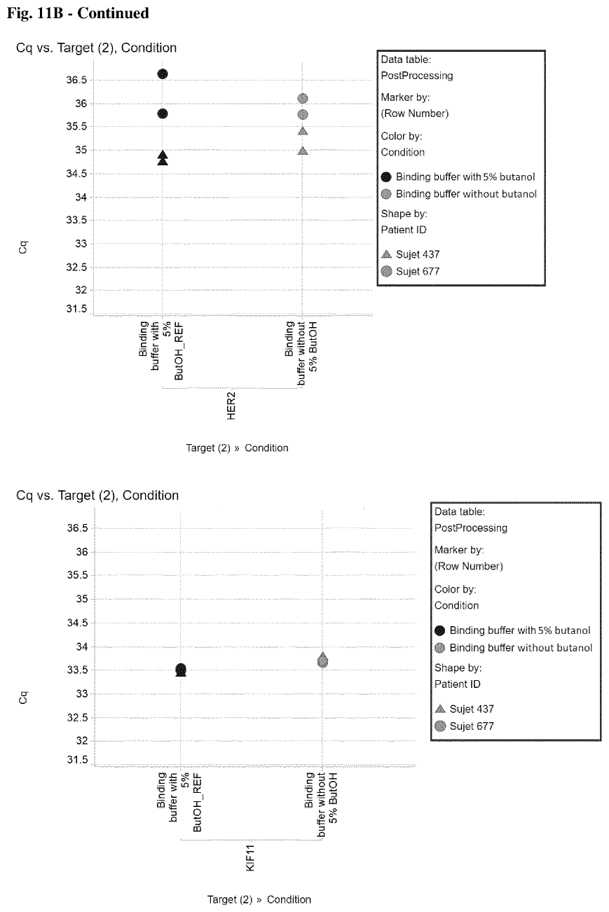

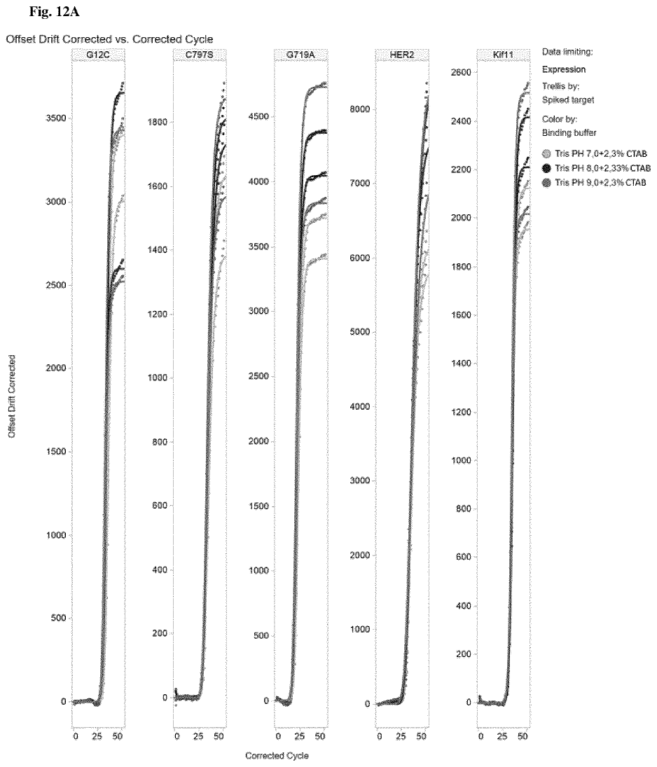

The results are summarized in FIGS. 11A and 11B.

The addition of 5% butanol had a slightly positive effect on the readout by on average 0.5 Cq. Although the effect was minute, it was observed uniformly over all different target sequences.

Thus, the QUATS-process of the invention was superior to the contemporaneous golden standard, with or without the addition of butanol. Nonetheless, due to its consistent behavior, addition of butanol may increase robustness of an assay.

### Example 3.5—Summary Conclusions

The present set of experiments demonstrated that free cations in the CB increased solubility, extraction efficiency and detection efficiency. Both monovalent or bivalent free cations could be used. The free cations improved the QUATS-process according to the invention over a wide range of concentrations from at least 50 mM to 400 mM. Moreover, the quaternary salt counter-anion had no effect on the influence of the free cations in the QUATS-process. Addition of butanol may increase the efficiency of the protocol even more.

### Example 4—the Effect of pH on the QUATS-Process

The inventors hypothesized that the particular QUATS of the present invention would have the ability to form micelles in a polar environment, in which the core is hydrophobic, the palisade layer of the QUATS is located on the inside of the micelles and the positively charged ammonium headgroup is located at the outside of the micelle (cationic micelle). The micelles of the invention would then form a complex between and with the negatively charged nucleic acids and the silica support. Example 2 demonstrated that the QUATS-process of the invention is highly efficient, and superior to the “Boom”-protocol.

In order to determine the effect of pH on the QUATS-process of the invention (see e.g. Vandeventer et al., 2012 J. Phys. Chem. B., 116:5661-5670), the inventors set out a series of experiments.

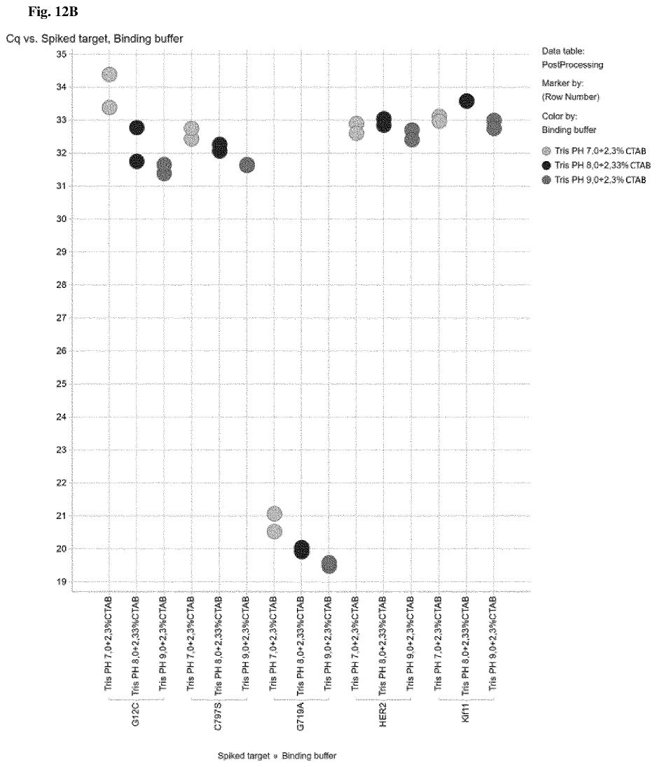

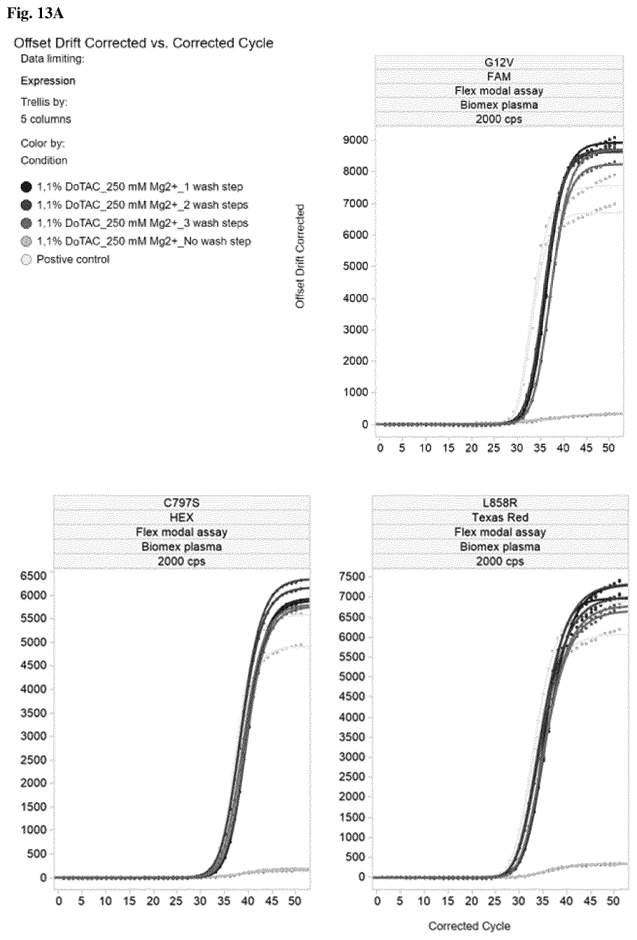

In essence, the experimental setup as described in Example 1 was used. In particular, QUATS-processes using a complexing buffer with 2.3% CTAB in Tris-buffer with pH 7, pH 8 and pH 9 were examined. The target sequences detected were KRAS G12C, EGFR C797S, EGFR G719A, HER2 and KIF11. Purification was performed on the bench (single plasma, PCR replicates) with amplification in a Bio-Rad thermal PCR cycler. The results are depicted in FIG. 12.

It can be seen that the QUATS-process functioned at all three pH values with about the same efficiency.

Lowering the pH outside this range of pH 7-9 resulted in vastly decreased extraction efficiencies (data not shown).

Hence, the QUATS-process was particularly efficient at pH 7 to pH 9.

### Example 5—the QUATS-Process Requires Less Wash Steps than the Gu-Based Protocol

The inventors hypothesized that the QUATS-process of the invention might require fewer washing steps than the comparator protocol using guanidinium for several reasons. The first being the change in ratio between sample:extraction compounds. Moreover, the QUATS-process uses less precarious compounds that might potentially interfere with post-purification steps. Hence, the QUATS-process might not require as much washes as the GU-based extraction protocol (3 wash steps with extensive amounts), which would save costs, reduce TAT, simplify the method, and would result in less loss of target material. The inventors set out to determine the number of wash steps for the QUATS-process.

In essence, Example 2.3 was repeated but now varying the number of wash steps, i.e. no wash step (negative control), 1 wash step, 2 wash steps, and 3 wash steps (current protocol). In this case, 90% ethanol was used as exemplary washing solution.

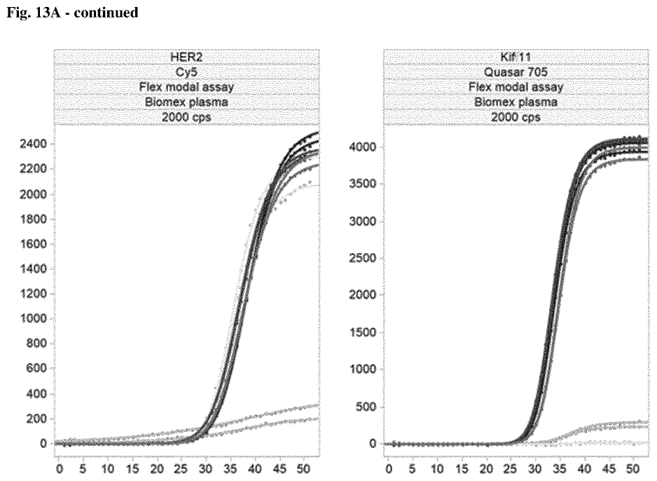

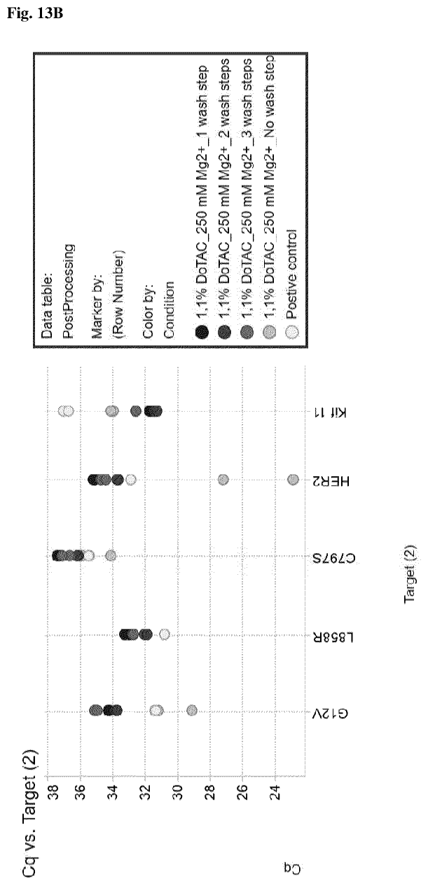

The results are summarized in FIG. 13.

As can be observed, one wash was necessary and sufficient in the QUATS-process of the invention. Indeed, 1 or 2 additional wash steps did not further improve the assay.

The one wash step experiment was repeated with varying concentrations of alcohol, i.e. 78%, 90% and 96% ethanol. All concentrations of alcohol tested resulted in good results, but 96% EtOH, which can be used of the shelf, seemed to be slightly better than the other concentrations, and also evaporated faster than lower percentage alcohols, resulting in less carryover in the eluate (results not shown).

### Example 6—the QUATS-Process' Elution Buffer is Compatible with Downstream Processes

The inventors hypothesized that the elution buffer in the QUATS-process of the invention should preferably comply with two requirements. On the one hand, the elution buffer should efficiently elute or release the nucleic acids from the silica support. On the other hand, the elution buffer should preferably be compatible with downstream processes, such as PCR and NGS, while not requiring further optimalisation steps, such as e.g. desalting or adjustment of the pH.

Accordingly, the inventors set out to design an elution buffer in the QUATS-process of the invention complying with these two requirements.

It was realized that PCR is carried out in a buffer that provides a suitable chemical environment for activity of DNA polymerase. The buffer pH is usually between 8.0 and 9.5 and is often stabilized by Tris-HCl, in which a pH 8.5 and higher Mg²⁺ is working better for the PCR. Contemporaneous protocols for eluting nucleic acids from silica membranes normally use Tris-HCl pH 8 buffers.

In essence, the experimental setup as described in Example 1 was used. In particular, the QUATS-process using a complexing buffer with DoTAC 1.1% at pH 8 and one wash with ethanol 96% was used, but distinguished by the use of six different types of elution buffers. In one arm, an elution buffer containing 3.8 mM of Mg²⁺ was tested at pH 8, pH 8.2, and pH 8.5. In a second arm, a buffer containing 4.6 mM of Mg²⁺ was tested at pH 8, pH 8.2, and pH 8.5. The sequences detected were EGFR G797S, KRAS G12V, HER2, KIF11 and EGFR L858R. Except for KIF11, 2300 copies were spiked into a sample.

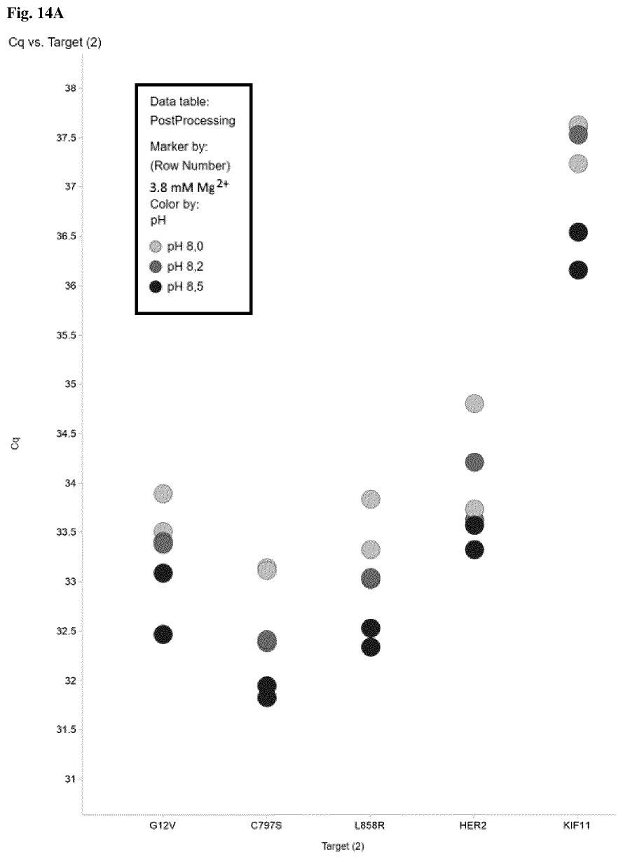

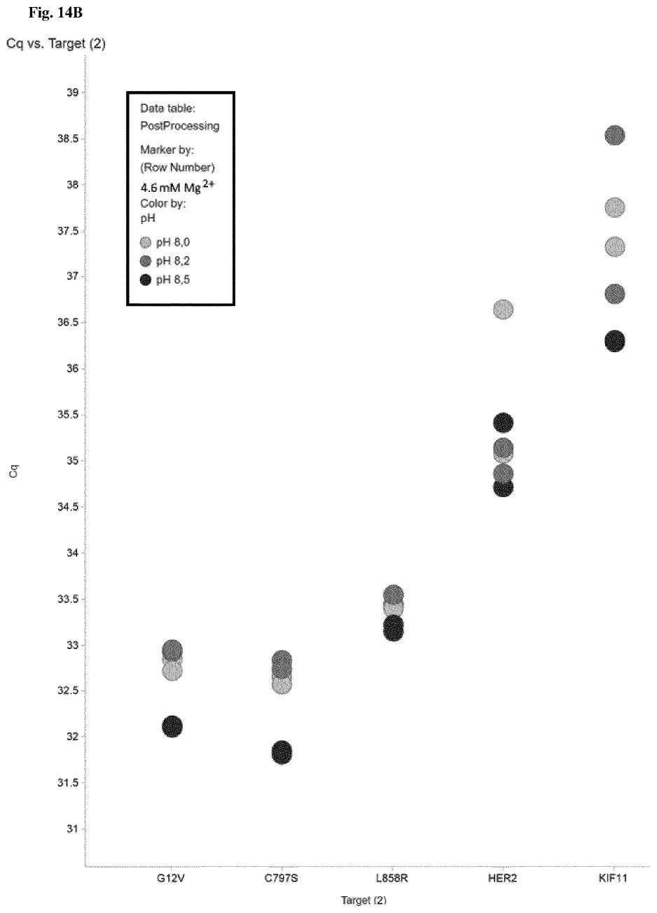

The results are depicted in FIG. 14.

It can be seen that all Mg²⁺ concentrations and all pH values, elute the nucleic acids efficiently and allow proficient processing of the purified nucleic acids without any further optimalisation steps. Nonetheless, pH 8.5 appears to be slightly better than the lower pH values at both Mg²⁺ concentrations, while higher Mg²⁺ concentrations appeared to be better than the lower Mg²⁺ concentrations, e.g. 4.6 mM Mg²⁺ appears to be slightly better than 3.8 mM Mg²⁺. The elution efficiency and processing compatibility of the elution buffer was confirmed at pH 8.4 (not shown).

Although the whole range of basic pH's was suitable from an elution and processing perspective, it was decided to use a pH of 8.4 from manufacturing, stability and safety perspective in the LoC.

### Example 7—Feasibility Performance of the QUATS-Process in Idylla™ Cartridges

In order to further examine the feasibility and superiority of the QUATS-process in LOC, the inventors tested the QUATS-process in both commercially available Idylla™ ctKRAS cartridges (standard cartridge), and cartridges in which the extraction buffer and process were replaced by the QUATS-based extraction buffer and process of the invention (test cartridge).

In particular, Limit-of-Detection (LoD) samples were made by adding a gBlock, i.e. synthetic double stranded DNA fragments of KRAS mutants (170 bps) in a background of normal human plasma matrix with fragmented cfDNA of on average 170 bps (AcroMetrix), which mimics the natural background of fragmented cell-free DNA in human plasma. Various KRAS mutants were divided in 4 different cocktails (cf. Table 2).

TABLE 2
KRAS mutants

| Cocktail 1 | Cocktail 2 | Cocktail 3 | Cocktail 4 |
|---|---|---|---|
| G12C | G12V | G12D | G13D |
| A146P | A146T | A146V | Q61R |
| A59E | A59T | Q61H | K117N2 |
| Q61K | K117N1 | | |
| | Q61L | | |

Samples were prepared at a medium input level (3000 cps/ml=10 ng/ml), which is representative for average plasma samples in respect of concentration cell-free DNA. The input level was formulated at 6 concentrations of a 2-fold serial dilution (see Table 3):

TABLE 3
Sample input levels

| medium input level | % AF* |
|---|---|
| 200 mutant cps/ml | 6.7 |
| 100 mutant cps/ml | 3.3 |
| 50 mutant cps/ml | 1.65 |
| 25 mutant cps/ml | 0.8 |
| 12.5 mutant cps/ml | 0.4 |
| 6.25 mutant cps/ml | 0.2 |

On*AF: Allelic Frequency

Next, all of the samples of Table 3 were lysed in the cartridges (standard cartridges & test cartridges) according to the standard, commercially available Idylla™ ctKRAS assay conditions, using proteinase K and HiFu (Biocartis NV). After lysis, the samples of Table 3 were each processed 6 times with both the contemporaneous Guanidinium-based extraction buffer of the Idylla™ ctKRAS assay (standard cartridge, cf. Example 2.1) and the complexing buffer of the invention (test cartridge). In the test cartridge, the complexing buffer comprised 50.00 mM Tris pH 8.0, 250.00 mM MgCl₂, 1.10% DoTAC and 5.00% butanol. Washing and elution buffers were identical in both the standard and the test cartridges, although the QUATS-process required only one washing step. The process parameters controlling the microfluidics of the cartridge systems were adjusted accordingly. The resulting, purified nucleic acids were assayed in the conventional ctKRAS qPCR disk for mutations in the KRAS biomarkers G12, G13, A59, Q61, K117 and A146, under standard conditions but for only 10 PCR cycles (linear phase).

The results are presented in Table 4, showing the estimated LoD values, based on % AF levels, at which a 100% hit rate is obtained (6/6 correct results).

TABLE 4

| Target | standard ctKRAS | | | | QUATS ctKRAS | | | |
|---|---|---|---|---|---|---|---|---|
| G12C | 0.8% | AF | 25 | cps/ml | 0.4% | AF | 12.5 | cps/ml |
| G12D | 3.3% | AF | 100 | cps/ml | 0.2% | AF | 6.25 | cps/ml |
| G12V | 0.8% | AF | 25 | cps/ml | 0.4% | AF | 12.5 | cps/ml |
| G13D | 6.7% | AF | 200 | cps/ml | ≤3.3% | AF | ≤100 | cps/ml |
| K117N1 | 1.67% | AF | 50 | cps/ml | 0.4% | AF | 12.5 | cps/ml |
| K117N2 | 1.67% | AF | 50 | cps/ml | ≤3.3% | AF | ≤100 | cps/ml |
| Q61H | 3.3% | AF | 100 | cps/ml | 0.4% | AF | 12.5 | cps/ml |
| Q61K | 3.3% | AF | 100 | cps/ml | 0.8% | AF | 25 | cps/ml |
| Q61L | >6.7% | AF | >200 | cps/ml | 0.8% | AF | 25 | cps/ml |
| Q61R | >6.7% | AF | >200 | cps/ml | 6.7% | AF | 200 | cps/ml |
| A146P | >6.7% | AF | >200 | cps/ml | 6.7% | AF | 200 | cps/ml |
| A146T | 6.7% | AF | 200 | cps/ml | 0.8% | AF | 25 | cps/ml |
| A146V | >6.7% | AF | >200 | cps/ml | ≤3.3% | AF | ≤100 | cps/ml |
| A59E | 6.7% | AF | 200 | cps/ml | 0.8% | AF | 25 | cps/ml |
| A59T | 6.7% | AF | 200 | cps/ml | ≤3.3% | AF | ≤100 | cps/ml |

In conclusion:

The feasibility data in this setup, i.e. the QUATS-process in a further conventional ctKRAS cartridge showed a decrease in error rate. No tracking errors were observed. This underscores the robustness of the QUATS-process.

Next, for all targets an improved performance (sensitivity) is observed of minimal 2-8 fold of the QUATS-process versus the GU-based extraction process, while still in the linear PCR phase.

An increased robustness and sensitivity will also improve the overall performance of a diagnostic assay.

1. A method for purifying nucleic acids from a solution comprising nucleic acids, the method comprising the steps of:
(i) complexing the nucleic acids in the solution to a silica support in the presence of a complexing buffer;
(ii) washing the nucleic acids of step (i) with an alcohol; and
(iii) eluting the nucleic acids of step (ii) with an elution buffer;
thereby purifying the nucleic acids;
wherein the complexing buffer comprises quaternary ammonium salt (QUATS) and free cation, and has a pH between 7-9,
wherein the QUATS comprises a quaternary ammonium cation having a nitrogen atom at the center and being represented by formula (I) 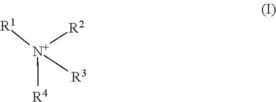
wherein each of R¹, R² and R³ are independently methyl groups (—CH₃) groups, and wherein R⁴ is an alkyl group (CₙH₂ₙ₊₁), with an even-numbered alkyl chain length,
wherein the free cation is chosen from the group consisting of Mg²⁺, Mn²⁺, Li⁺, Na⁺, Zn²⁺, K⁺ and Ca²⁺, and
wherein the concentration of the free cation is between 50 mM and 0.5 M.

2. The method for purifying nucleic acids from a solution according to claim 1, wherein the QUATS:
has a counter-anion chosen from Cl⁻ and Br⁻, and/or
is present in a concentration (mass/vol) between 0.5% to 7%, and/or
is chosen from the group consisting of:
octyl-trimethyl-ammonium bromide (OTAB);
octyl-trimethyl-ammonium chloride (OTAC);
decyl-trimethyl-ammonium bromide (DTAB);
decyl-trimethyl-ammonium chloride (DTAC);
dodecyl-trimethyl-ammonium bromide (DoTAB);
dodecyl-trimethyl-ammonium chloride (DoTAC);
tetradecyl-trimethyl-ammonium bromide (TTAB);
tetradecyl-trimethyl-ammonium chloride (TTAC);
hexacetyl-trimethyl-ammonium bromide (CTAB);
hexacetyl-trimethyl-ammonium chloride (CTAC);
octadecyl-trimethyl-ammonium bromide (OctMAB); and
octadecyl-trimethyl-ammonium chloride (OctMAC).

3. The method for purifying nucleic acids from a solution according to claim 1, wherein the complexing buffer comprises free cations in a concentration of about 50 mM, about 100 mM, about 150 mM, about 175 mM, about 200 mM, about 225 mM, about 250 mM, about 275 mM, about 300 mM, about 325 mM, about 350 mM, about 400 mM, about 450 mM, or about 0.5 M.

4. The method for purifying nucleic acids from a solution according to claim 1, wherein the silica support is chosen from the group consisting of glass particles, glass powder, silica particles, glass microfibers, diatomaceous earth, silica sand, silica gel, silicon carbide (SiC), silica membranes, silica impregnated or coated filters, silica coated magnetic beads and mixtures thereof.

5. The method for purifying nucleic acids from a solution according to claim 1, wherein the alcohol;
is chosen from methanol, propan-1-ol, isobutanol, sec-butanol, tert-butyl-alcohol, ethanol, butanol and isopropanol, and/or
is present in concentration of between 70% to 100%.

6. The method for purifying nucleic acids from a solution according to claim 1, wherein the elution buffer:
has a pH between 8 and 9, and/or
comprises:
Tris-HCl at a concentration between 0.01 M and 0.05 M;
MgCl₂ at a concentration between 3 mM and 11 mM; and
KCl at a concentration between 40 mM to 150 mM.

7. The method for purifying nucleic acids from a solution according to claim 1, wherein the nucleic acids are derived from a tissue sample.

8. The method for purifying nucleic acids from a solution according to claim 1, wherein the nucleic acid is DNA.

9. The method for purifying nucleic acids from a solution according to claim 1, wherein the nucleic acid is purified to at least 60% purity.

10. The method for purifying nucleic acids from a solution according to claim 1, wherein step (i) is preceded by:
a lysis step, and/or
a protease treatment, and/or
a liquefaction step.

11. The method for purifying nucleic acids from a solution according to claim 1, wherein the method is performed inside of a cartridge.

12. A cartridge configured to purify nucleic acids from a solution, the cartridge comprising at least three containers for storing liquids, at least a sample chamber adapted for receiving a sample, and a processing chamber comprising a silica support,
wherein the containers are in fluid connection with the sample chamber and/or the processing chamber, and wherein:
at least one container containing a complexing buffer;
at least one container containing an alcohol; and
at least one container containing an elution buffer;
wherein the complexing buffer comprises quaternary ammonium salts (QUATS) and free cation and has a pH between 7-9,
wherein the QUATS comprises a quaternary ammonium cation having a nitrogen atom at the center and being represented by formula (I) 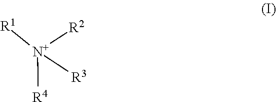
wherein each of R¹, R² and R³ are independently methyl groups (—CH₃) groups, and wherein R⁴ is an alkyl group (CₙH₂ₙ₊₁), with an even-numbered alkyl chain length,
wherein the free cation is chosen from the group consisting of Mg²⁺, Mn²⁺, Li⁺, Na⁺, Zn²⁺, K⁺ and Ca²⁺, and
wherein the concentration of the free cation is between 50 mM and 0.5 M.

13. A method for purifying nucleic acids from a solution comprising nucleic acids, the method comprising the steps of:
(i) complexing the nucleic acids in the solution to a silica support in the presence of a complexing buffer;
(ii) washing the nucleic acids of step (i) with an alcohol; and
(iii) eluting the nucleic acids of step (ii) with an elution buffer;
thereby purifying the nucleic acids;
wherein the complexing buffer comprises quaternary ammonium salt (QUATS) and free cation, and has a pH between 7-9,
wherein the QUATS comprises a quaternary ammonium cation having a nitrogen atom at the center and being represented by formula (I) 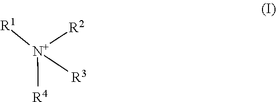
wherein each of R¹, R² and R³ are independently methyl groups (—CH₃) groups, and wherein R⁴ is an alkyl group (CₙH₂ₙ₊₁), with an even-numbered alkyl chain length, wherein the free cation is chosen from the group consisting of Mg²⁺, Mn²⁺, Li⁺, Na⁺, Zn²⁺, K⁺ and Ca²⁺, and
wherein the concentration of the free cation is between 50 mM and 0.5 M,
wherein the method is carried out with the cartridge according to claim 12.

14. A complexing buffer comprising quaternary ammonium salt (QUATS) and free cation, wherein the complexing buffer has a pH between 7-9,
wherein the QUATS comprises a quaternary ammonium cation having a nitrogen atom at the center and being represented by formula (I) 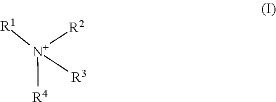
wherein each of R¹, R² and R³ are independently methyl groups (—CH₃) groups, and wherein R⁴ is an alkyl group (CₙH₂ₙ₊₁), with an even-numbered alkyl chain length,
wherein the free cation is chosen from the group consisting of Mg²⁺, Mn²⁺, Li⁺, Na⁺, Zn²⁺, K⁺ and Ca²⁺, and
wherein the concentration of the free cations is between 50 mM and 0.5 M.
